(19) United States
(12) Patent Application Publication
(10) Pub. No.: US 2009/0253154 A1
(43) Pub. Date: Oct. 8, 2009

(54) Blood and saliva test for detection of delayed food allergy and intolerance against modified foods

(75) Inventor: Aristo Vojdani, Beverly Hills, CA (US)

Correspondence Address: Cislo & Thomas LLP, 1333 2nd Street, Suite #500, Santa Monica, CA, 90401-4110, US

(73) Assignee: Immunosciences Lab., Inc., Beverly Hills, CA (US)

(21) Appl. No.: 12/061,314

(22) Filed: Apr. 2, 2008

(51) Int. Cl. G01N 33/53; G01N 33/563

(52) U.S. Cl. 435/7.92; 436/513

(57) ABSTRACT

A method for determining the presence of delayed food allergy and intolerance against antigens extracted from modified foods. The method includes determining a level of antibodies against a modified dietary food antigen in blood and mucosal samples from the patient and comparing the level with normal levels of the antibodies. Dietary antigens that were tested include milk and modified milk products; eggs and modified egg products; meat and modified meat products; fish, mollusks, and crustaceans and their modified products; oils, fats and their modified products; grains and modified grain products; pulses, seeds kernels, nuts and their modified products; vegetables and modified vegetable products; fruits and modified fruit products; sugar, modified sugar products, modified chocolate products and confectionery; and spices and their modified forms.

### BACKGROUND OF THE INVENTION

1. Field of the Invention

The invention relates to an immunoassay for delayed food allergy and intolerance against modified foods.

2. Description of the Related Art

Food allergy has become a problem that concerns many clinicians. Adverse reactions to foods in which the pathogenesis involves an immunological response to food components are appropriately called food-hypersensitivity reactions. This term is considered to be synonymous with “food allergy.” This adverse immune reaction to food proteins affects as many as 6% of young children and 3-4% of adults (Sicherer and Sampson, 2006). However, in a study using double-blind placebo-controlled food challenge, 39% of participants showed hypersensitivity to food antigens (Bock and Atkins, 1990).

Immune-mediated adverse reactions to foods can be divided into distinct clinicopathologic entities based on presentation (immediate or delayed), target organ specificity, and pathogenic mechanisms. By far, the most recognized reactions are IgE mediated and dependent on activation of mast cells in specific tissues. Such reactions are immediate and in severe cases may be life-threatening (Sampson et al., 1999, 2004).

Immediate reactions to foods can involve one or more target systems, including the skin, respiratory tract, gastrointestinal, mucosal, and cardiovascular system (Sampson et al., 1992; Eigenmann et al., 1998; Bischoff and Crowe, 2005).

Careful clinical observation has made it possible to document that the signs and symptoms initially follow a pattern reflecting the sites of initial exposure to the incriminated food. Thus, oropharyngeal reactions are frequently reported first, followed by gastrointestinal responses, and then involvement of the skin and respiratory tract (Gordon, 2003).

Unlike the immediate effects of IgE-mediated allergy, the IgG, IgM and IgA-mediated food allergy and intolerance reactions can take several days to appear. Levels of IgG, IgM and IgA antibodies in the blood against different food antigens have been used for demonstration of delayed food allergy and intolerance reactions (Hvatum et al., 1992; Barnes, 1995). Therefore, raised serum or plasma IgG, IgM and IgA levels of food-specific antibodies are often associated with delayed food allergies and intolerance. This classification of immune mechanisms related to dietary proteins and peptides is shown in Table 1.

TABLE 1
Classification of immune mechanisms
to dietary proteins and peptides

| | |
|---|---|
| Gastrointestinal intolerance | IgA and IgM mediated |
| Immediate type hypersensitivity | IgE mediated |
| Type-I | |
| Delayed immune reaction | |
| Type-II Cytolytic | IgG and IgA mediated |
| Type-III | IgG and IgA immune |
| | complex mediated |
| Delayed hypersensitivity | Cell mediated with involvement of |
| Type-IV | cytokines |

However, measurement of IgG, IgM or IgA in the blood may miss abnormal immune reaction to many food antigens. In one instance, it is known that oral or intragastric administration of antigens results in salivary IgA production, but not in any antibody production in serum (Walker and Isselbacher, 1977; Challacombe, 1987; Kanda et al., 2001).

### Manifestation of IgA Antibodies in Secretions

The deposition of antigens in the gut has been shown to lead to the production of IgA antibodies in secretion at sites distant from the gut, such as colostrums, lacrimal and salivary secretions (Brandtzaeg et al., 1970; Brandtzaeg, 2003, 2007).

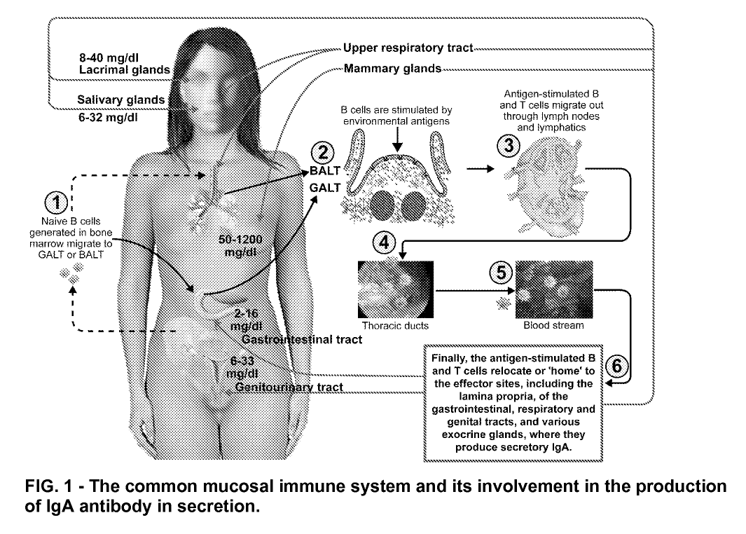

It can be concluded, therefore, that the secretory immune system can be stimulated centrally and that precursors of IgA-producing_cells migrate from the gut-associated lymphoid tissue to several_secretory sites, as well as to the lamina propria of the gut_itself. Therefore, if antigens are injected into the submucosal tissues, they are likely to induce serum IgG antibodies_as well as secretory IgA antibodies in saliva. However, if it_is applied topically to the skin or to the intraepithelial tissue, the resultant main product is only secretory IgA, which is detected_in saliva (see FIGS. 1-3).

Based on this mechanism of action, saliva is a source of body fluid for detection of an immune response to bacterial, food, and other antigens present in the oral cavity and gastrointestinal tract. Indeed, salivary antibody induction has been widely used as a model system to study secretory responses to ingested material, primarily because saliva is an easy secretion to collect and analyze. Indeed, saliva, as a diagnostic fluid, is certainly preferable to blood in the pharmacological monitoring of patients with chronic therapies (Jertborn et al., 1986; Taubman and Smith, 1993; Nogueira et al., 2005; Hakeem et al., 1992; Al-Bayaty et al, 1989; Rumbo et al., 1998).

Besides its protective and lubricating properties, saliva meets the demand for inexpensive and easy-to-use diagnostic aids for oral and systemic diseases.

As mentioned previously, food allergy has become a problem that concerns many clinicians. Adverse reactions to foods in which the pathogenesis involves an immunological response to food components are appropriately called food-hypersensitivity reactions. Based on this, we have developed a test using blood as well as saliva that will accurately inform a physician of clinical conditions and diagnose patients who may suffer from food allergies or food intolerance. The test measures antibody levels to dietary antigens or peptides (Hakeem et al., 1992; Brandtzaeg, 2007).

The antibodies present in saliva are IgA (90%) and IgM (10%). For detection of these antibodies, saliva can be a source of body fluid for the measurement of immune response to dietary antigens present in the nasopharyngeal cavity and gastrointestinal tract (Czerinsky et al., 1987; Kunishawa and Kiyono, 2005).

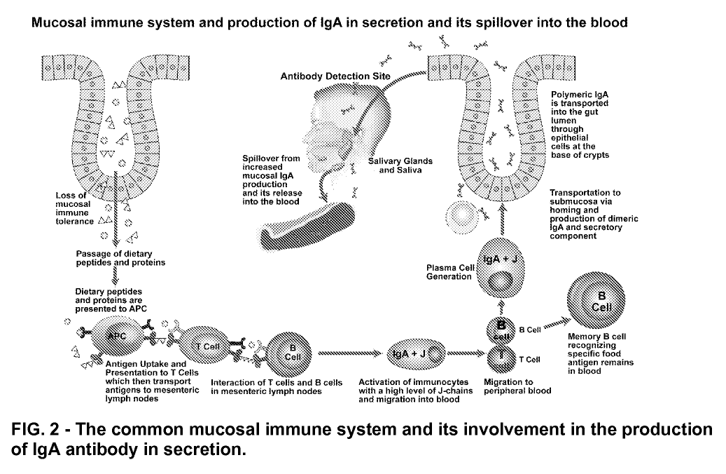

Therefore, mucosal immune system reaction to dietary proteins and peptides can result in production of IgA antibody in secretion. The immunological mechanism behind this IgA antibody production in saliva and its spillover into the blood is shown in FIGS. 1 and 2. FIGS. 1 and 2 show the common mucosal immune system and its involvement in the production of IgA antibody in secretion. Naïve B cells generated in the bone marrow migrate to the inductive sites of mucosal immunity represented by the gut-associated lymphoid tissues (GALT; Peyer's patches and lymphoid follicles of the large bowel) or bronchus-associated lymphoid tissues (BALT) (1), where they are stimulated by antigens taken up and presented by antigen-presenting cells and cognate T-helper cells (2). Antigen-stimulated B and T cells migrate out through the draining lymph nodes and lymphatics (3), enter the blood stream (4) (5), and finally relocate or ‘home’ to the effector sites, including the lamina propria, of the gastrointestinal, respiratory and genital tracts, and various exocrine glands, where they secrete a significant amount of IgA antibody (6).

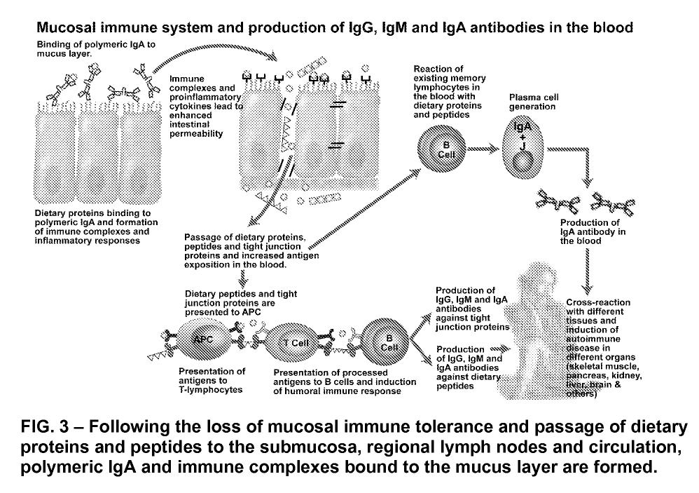

However, if the patient suffers from enhanced intestinal permeability due to mucosal inflammation, dietary proteins and peptides may pass through the tight junctions with an ensuing entry of dietary peptides and tight junction proteins into the circulation (Sicherer and Sampson, 2006; Faria and Weiner, 2005; Adams and Eksteen, 2006; Fasano and Shea-Donohue, 2005). The presentation of these antigens by antigen-presenting cells to memory B cells results in the production of IgA antibody against dietary proteins and peptides in the blood within a short period of time (for example, 7 days). Further presentation of antigens to T cells and B cells results in IgG, IgM and IgA antibodies against dietary proteins and peptides in the blood, which may take a longer period of time (for example, 15 days). The possible immunological mechanism behind this mucosal immune response to dietary proteins and peptides, enhanced intestinal permeability, and production of IgG, IgM and IgA antibodies in blood is shown in FIG. 3.

FIG. 3 shows how, following the loss of mucosal immune tolerance and passage of dietary proteins and peptides to the submucosa, regional lymph nodes and circulation, polymeric IgA and immune complexes bound to the mucus layer are formed. Immune complexes and inflammatory cytokines can lead to enhanced gut permeability and the entry of dietary proteins and peptides into the circulation. Of these antigens and peptides are presented to memory B lymphocytes, generated due to prior exposure to the same antigen, then plasma cells with IgA+J chain are formed to produce IgA antibodies in the blood within 7 days. However, if these antigens and peptides are presented to antigen presenting cells, T cells and B cells, then plasma cell formation can result in the production of IgG, IgM and IgA antibodies against dietary proteins and peptides in the blood within 15 days.

Why are Purification and Characterization of Dietary Proteins and Peptides from Processed Foods Essential for Reproducibility of IgA+IgM in Saliva and IgG, IgM and IgA in Blood?

Processed foods and their ingredients are subjected to a variety of processing conditions, which may cause alterations in immunodominant epitopes, potentially affecting allergenic properties. This processing may destroy existing epitopes on a protein or may cause new ones to be formed (neoallergen formation) as a result of change in protein conformation. Neoallergen formation has been known for at least three decades (Spies, 1974); it may be part of the reason why some individuals can tolerate a raw food or food ingredient but will react to the same food when it is processed. Recently, studies have found neoallergens from pecans (Malanin et al., 1995) and wheat flour (Leduc et al., 2003). Processing methods are more commonly associated with decreased allergenicity (i.e., pollen-related fresh fruit and vegetable food allergens upon heating) or as having no significant effect (i.e., heat-stable allergens from shrimp upon heating). Typically, conformational epitopes are seen as being more susceptible to processing-induced destruction than the linear epitopes on the same allergen. The latter are more likely to be altered if hydrolyzed. Linear epitopes may also be chemically modified during food processing, as well as intentionally changed by mutations introduced through genetic engineering. The different types of food processing includes thermal as well as non-thermal treatments, and each type of process may have a different effect on epitopes. In evaluating allergen stability, then, the different effects of individual treatments must be considered carefully. Thermal processing may be done by dry heat (e.g., oven roasting, oil roasting, infra-red heating, ohmic heating) or wet heat (e.g., boiling, microwave cooking, pressure cooking, autoclaving, extrusion, blanching, steaming). Non-thermal treatments include irradiation, soaking, germination, milling, fermentation, high-pressure processing, dehulling and dehusking, and grinding. Processing may affect food in a manner that may induce the masking or unmasking of allergenic epitopes, thereby enhancing or reducing allergen recognition and potentially altering the allergenicity of the offending food. Food processing may alter an epitope's protein structure, leading to destruction, modification, masking or unmasking, which may in turn lead to decreasing, increasing, or having no effect on allergenicity. One study (Sanchez and Fremont, 2003) extensively reviewed the effects of food processing on the structural stability of food allergens and concluded that effects of protein-protein interactions (especially aggregation) are virtually unknown. The examples that follow illustrate the diversity and complexity of issues involved in determining the effectiveness and limitations of food processing methods as a tool in understanding neoallergenicity. In relation to common processing methods, including mechanical, enzymatic, heating, drying, peeling, pulping, blanching, mashing, pasteurization and multiple-treatment effects on the allergenicity of processed food antigens, all the published articles dealt with immediate hypersensitivity reaction which is IgE mediated (Sathe et al., 2005), and none dealt with delayed immune reaction to processed food antigens. This sampling of articles also illustrates that in a majority of cases some of the technological processing treatments not only maintained their antigenicity and allergenicity but also induced the modification and introduction of neoantigens.

A study conducted by Leduc et al., in 2003 reported a case of food allergy to a wheat isolate used in the meat industry, but without any allergic reaction to native wheat flour. A 24-year-old woman had 2 episodes of angioedema with generalized urticaria less than 30 minutes after the ingestion of a sausage and pork pie. She had regularly eaten bread and pork-based products without any reaction. Realistic skin prick test responses to the fresh meat pie components (crust, crumb, meat, and sausage) were positive (i.e., wheal responses of 5 to 10 mm). The manufacturer informed investigators that the pork pie contained a wheat isolate. Native skin prick test reactions to this wheat isolate (isolate A) were positive, with 19-mm and 8-mm wheal responses (raw and cooked, respectively). Three wheat flour isolates were studied: wheat isolates A and B from 2 different producers were obtained by means of acid treatment and heat, and wheat isolate C was obtained by means of enzymatic treatment. Wheat isolates were extracted for 2 hours at a 1:20 w/v ratio in Coco buffer (0.55% NaHCO₃, 1% NaCl). Immunoblotting of the antigen with the patient's serum but not controls showed strong IgE response to wheat isolate, but no specific IgE antibodies to wheat flour fractions were detected. IgE inhibition assays by means of ELISA showed that the 3 isolates were able to inhibit patient's serum IgE binding to adsorbed wheat isolate. This absence of ELISA inhibition of wheat isolates by means of gliadin fractions could argue for a neoallergenicity. Deamidation of the gluten fraction by means of technologic processes (chemical or enzymatic) could have induced the exposure of cryptic allergenic epitopes or the formation of new allergenic epitopes (Leduc et al., 2003). Similar to this, a case of contact urticaria has been recently attributed to hydrolyzed wheat in cosmetics combined with a generalized urticaria induced with the ingestion of sausages with lentils and a French cassoulet (Pecquet et al., 2002). It was concluded that wheat isolates should be tested when a food allergy to finished food is suspected.

Raw peanut extracts are used in the majority of current allergy tests, even though persons rarely ingest raw peanuts. For this reason, the biochemical effects of roasting on the allergenic properties of peanut proteins was studied (Maleki et al, 2000). Competitive inhibition ELISA was used to compare the IgE-binding properties of roasted and raw peanut extracts. The allergic properties were measured by using ELISA, digestion by gastric secretions, and stability of the proteins to heat and degradation. It was shown that roasted peanuts from two different sources bound IgE from patients with peanut allergy at approximately 90-fold higher levels than the raw peanuts from the same peanut cultivars. The purified major allergens Ara h 1 and Ara h 2 were subjected to the Maillard reaction in vitro and compared with corresponding unreacted samples for allergenic properties. Ara h 1 and Ara h 2 bound higher levels of IgE and were more resistant to heat and digestion by gastrointestinal enzymes once they had undergone the Maillard reaction. The study concluded that thermal processing may play an important role in enhancing the allergenic properties of peanuts and that the protein modifications made by the Maillard reaction contribute to this effect. A different study (Cheung and Champagne, 2001) reported an association between advanced glycation end product adducts and increased IgE binding with roasted peanuts, but not with raw peanuts.

The purposes of yet another study (Codina et al., 1998) were (1) to evaluate the allergenicity of fresh and stored soybean hulls and (2) to ascertain whether heat alters the allergenicity of stored soybean hulls. During the process of harvest, transport and storage, microbial and mold contamination can raise the temperature of soybeans to 75° C. or higher. Allergen extracts were prepared from (1) stored soybean hulls, (2) fresh soybean hulls and (3) stored soybean hulls heated to 37° C. (E1), 55° C. (E2) and 80° C. (E3) or kept at room temperature (E4) for 16 h. Individual serum from 68 soybean asthmatic (SA) subjects, 30 nonallergic subjects and two serum pools made from 4 SA sera and 4 sera from asthmatics not sensitive to soybean were studied. All sera and serum pools were assayed for content of specific IgE (radioallergosorbent test) and IgG4 (ELISA). The following additional studies were done for extracts E1-E4: (1) SDS-PAGE, (2) SDS-PAGE/Western blot for specific IgE against fresh raw and heated soybean antigens Test results demonstrated a reduced binding of specific IgE and IgG4 to fresh soybean hull extract compared to stored soybean hull extract, and an increased binding for heated extracts (E1-E3) compared to unheated ones (E4). Moreover, there was an increase in potency for IgE and IgG4 bindings for the heated (E1-E3) compared to unheated (E4) extract, as measured by the amount of protein to produce 50% inhibition. Using SDS-PAGE, a new protein band MW of 15.3 kDa appeared for heated soy. These results demonstrate that soybean hull allergenicity is affected by heat, and suggest that the heat generated during storage and transport of soybeans could generate 2 new allergen determinants or increases in epitope exposure as a result of conformational changes.

This next study investigates anaphylactic reaction caused by neoallergens in heated pecan nut (Malanin et al., 1995). An allergic girl experienced type-I IgE-mediated reaction after eating cookies containing pecan nuts. Investigation revealed that she developed IgE antibodies made specifically against allergenic determinants present in aged or heated pecan nuts but not in fresh pecans. The molecular size for this neoallergenic determinant was determined to be 15 kDa. It was concluded that neoallergens appearing during the heating or storing of foods are important in some individuals with anaphylactic reaction.

The impact of γ-irradiation and thermal processing on the antigenicity of almond, cashew nut and walnut proteins was the focus of this study (Su et al., 2004). Whole unprocessed almonds, cashew nuts and walnuts were each subjected to _-irradiation followed by heat processing including autoclaving, dry roasting, blanching, oil roasting and microwave heating. Rabbit polyclonal antibodies were raised against each major protein isolated from defatted, but not subjected to _-irradiation and/or any thermal processing, almond, cashew nut and walnut flours. Immunoreactivity of almond, cashew nut and walnut proteins was determined by inhibition enzyme-linked immunosorbent assay (ELISA) and Western blotting. ELISAs and Western blotting experiments indicated that almond, cashew nut and walnut proteins exposed to various thermal treatments and _-irradiation remained antigenically stable.

This particular team studied the retention of allergenicity by soy sauce through the fermentation/production process (Hefle et al., 2005). Soy allergy is one of the most prominent allergies in the worldwide population. The vast majority of soy sauces are produced through the fermentation of soy and wheat. Some soy sauce manufacturers tell finished food product processors that the fermentation process destroys the allergenicity of their soy and wheat fermentation ingredients. This has not proven to be the case by scientific experimentation, so the risk of reaction from soy sauce ingestion among soy-allergic and wheat-allergic/celiac patients is unknown. Indeed, when ten soy sauces were evaluated by ELISA and RAST inhibition, it was shown that soy sauces made by the fermentation of soy protein can retain their antigenicity and allergenicity through the fermentation process. Therefore, soy-allergic patients should continue to be counseled to avoid soy sauce.

It is often thought that thermal processing should decrease allergenicity (Malanin et al., 1995), since heating or cooking normally causes a catastrophic disruption of proteins structure. Yet the first properly reported case of food allergy (Prausnitz and Kustner, 1921) was an example of the exact opposite—a food allergy in which the patient was allergic to cooked fish protein, but not to raw fish protein. This is a neat irony, for, since that time, there have been few reported cases of food allergy restricted to cooked foods. But there should be no surprise in the often repeated finding that heat treatment does not do much to reduce allergenic risk. There are many ways in which the antigenicity of proteins can be enhanced during thermal processing, especially when this processing takes place in the complex milieu of a food, with so many other ingredients available to participate in complex physical and chemical reactions (Davis and Williams, 1998).

Far from being a general way to decrease allergenic risk, thermal processsing is as likely to increase allergenicity as to reduce it, through the introduction of neoantigens. These changes are highly complex and not easily predictable, but there are a number of chemical pathways that lead to distinct patterns of modification. Perhaps the most important of these is through the reaction of protein amino groups with sugars, leading to an impressive cocktail of advanced glycation end-product (AGE)-modified protein derivatives. These are antigenic and many of the important neoantigens found in cooked or stored foods are probably such Maillard reaction products (Davis, 2001). The formation of AGE during food processing through this mechanism of action can have a potent impact in tissue inflammation, a process linked to diverse biological settings such as diabetes, metabolic syndrome, renal failure and aging (Ramasamy et al., 2005, Bengmark, 2007).

During the processing of food many lipids may become oxidized (Doke et al., 1989). Auto-oxidized lipids could interact with various proteins and form new antigenic materials. The allergenicity of proteins interacted with oxidized lipids was examined by enzyme-linked immunosorbent assay (ELISA) using sera from soybean-sensitive individuals. Though oxidized soybean oil did not show any allergenicity, the IgE titer of sera from soybean-sensitive patients was greatly increased when oxidized soybean oil was incubated with soybean 2S-globulin. The IgE titer of patient sera became higher when greater amounts of oxidized soybean oil were used. These results clearly show that proteins interacted with oxidized lipids are allergenic to soybean-sensitive patients.

In food anaphylaxis cases, proper identification of the allergen responsible for the anaphylaxis is vital to prevent life-threatening reactions (Rosen et al., 1994). The prick skin test (PST) with commercially prepared allergens has been the hallmark procedure for detecting IgE antibodies against food allergens. This article describes 22 patients from two large pediatric and adult allergy practices who have a clinical history of anaphylaxis to foods with negative skin test results to commercial food extracts but positive test results to natural food extracts used for skin testing. One possible reason for the negative results to commercial food and positive results to natural food may be that certain specific allergens are lost during the processing of the commercial food extracts. Cooking food may in some cases destroy allergens, although there are also cases (e.g., shrimp) wherein allergenicity is maintained or even increased. One patient, when tested against shrimp, tested negative with raw shrimp but unequivocally positive with steamed shrimp. The PST was repeated twice on the patient with the same results. It appears that instead of destroying all the allergens, cooking the shrimp may have produced new allergens not present in the raw shrimp, perhaps by exposing allergenic epitopes already present in the shrimp or by altering existing allergenic proteins. This is the best example of how an individual may appear allergy-free when tested with raw foods, but in reality may be highly allergic to processed foods.

These examples clearly indicate that food processing in some cases may decrease allergenic risk, but in a majority of cases maintains or even enhances allergenicity, and in particular, antigenicity. Protein conformation (for example, by heating) leads to the generation of new epitopes on proteins called neoallergens. If these new epitopes or neoallergens can induce new IgE response, then they can also induce new IgG, IgM and IgA responses.

### SUMMARY OF THE INVENTION

For many commercial suppliers of food antigens or allergens, almost all food antigens and allergens are prepared from raw food rather than processed food. Because it is well accepted that a majority of people do not typically consume uncooked food, in this application we sought to prepare extracts from processed foods and compare them to extracts from raw food samples. In reality, more than 95% of the population consumes modified foods rather than raw foods. Since all examples of new allergenicity to food antigens shown here and many others published in scientific journals deal only with IgE mediated or type-I allergic reaction, we decided to extend this investigation to non-IgE mediated antibodies produced against extracts prepared from modified foods purchased from supermarkets or restaurants. This includes IgA and IgM in saliva, as well as IgG, IgM and IgA in blood. The measurement of IgA and IgM in saliva and IgG, IgM and IgA in blood against modified food antigens results in an enhancement in the detection of delayed food sensitivities or intolerance that would not be possible by merely measuring these same antibodies against antigens prepared from raw or unprocessed foods alone.

Disclosed herein is a method of measuring IgA and IgM in saliva, as well as IgG, IgM and IgA in blood against different modified food antigens and peptides for use in determining food allergy and food intolerance.

A method for determining the presence of food allergy or food intolerance in a patient includes (a) determining a level of antibodies against a modified dietary antigen present in the food in a blood or saliva sample from the patient; and (b) comparing the level determined in step (a) with normal levels of the antibodies in said sample.

The possible outcomes for the comparison include (i) lower than normal levels or about normal levels of dietary antigen antibodies indicate optimal conditions; and (ii) higher than normal levels of dietary antigen antibodies indicate a food allergy or food intolerance.

Further objects, features and other advantages of the preferred embodiments become apparent from the ensuing detailed description, considered together with the appended figures.

### BRIEF DESCRIPTION OF THE DRAWINGS

FIG. 1
The common mucosal immune system and its involvement in the production of IgA antibody in secretion.

FIG. 2
The mucosal immune system, the production of IgA in secretion, and its spillover into the blood.

FIG. 3
Following the loss of mucosal immune tolerance and passage of dietary proteins and peptides to the submucosa, regional lymph nodes and circulation, polymeric IgA and immune complexes bound to the mucus layer are formed.

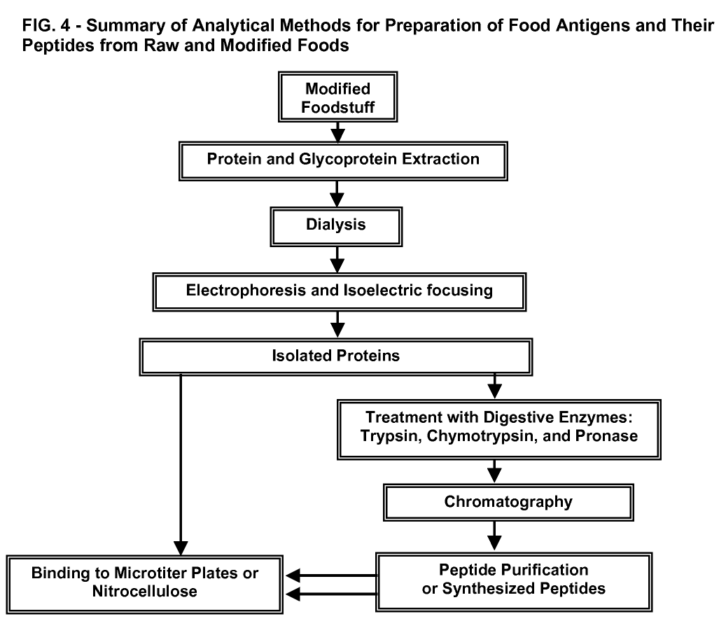

FIG. 4
Summary of analytical methods for preparation of food peptides and their antigens from raw and modified foods.

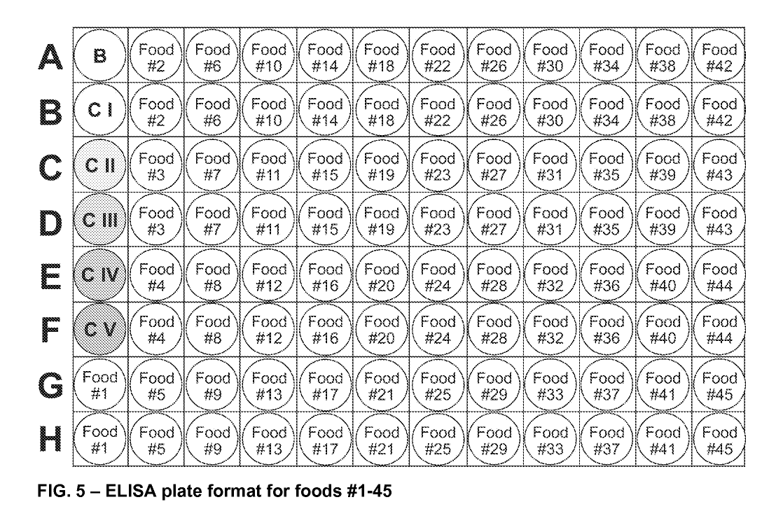

FIG. 5
ELISA plate format for foods #1-45.

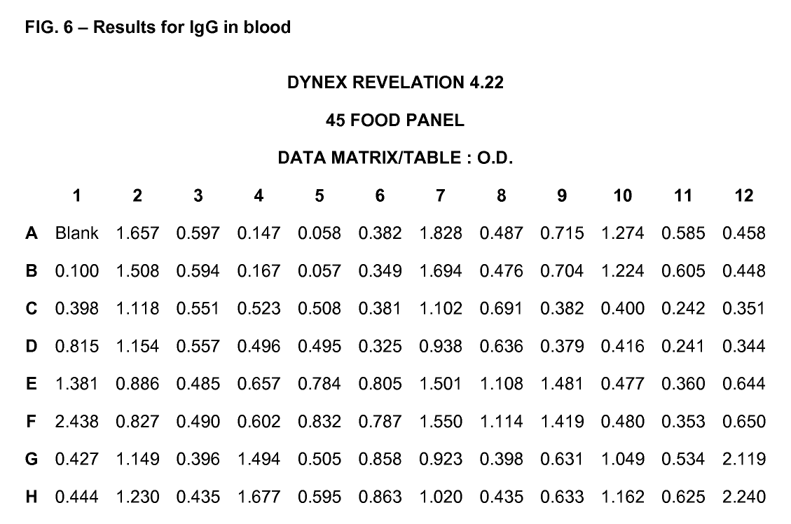

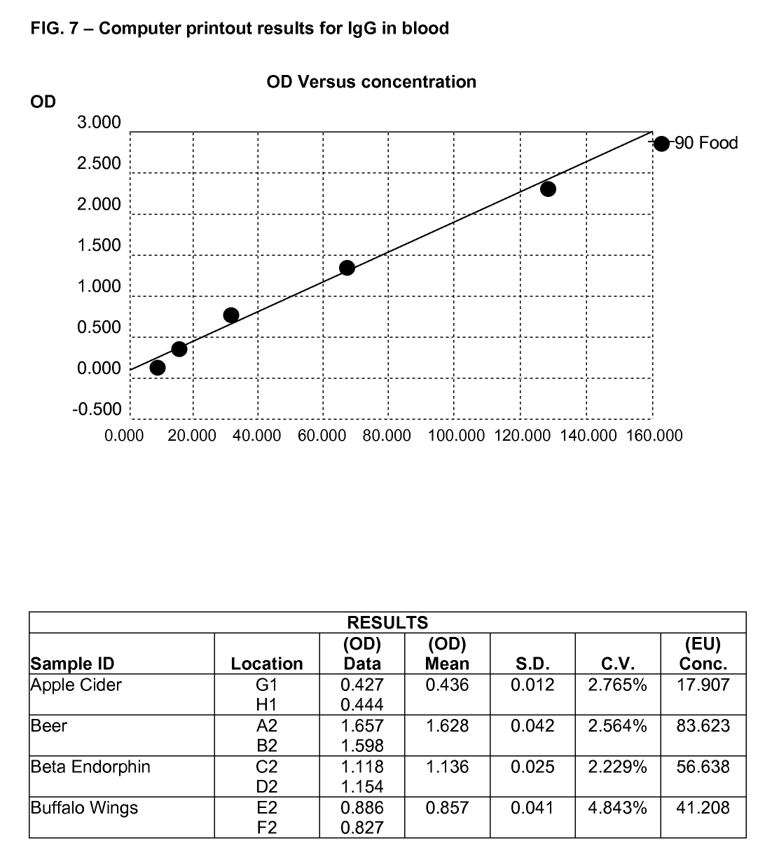

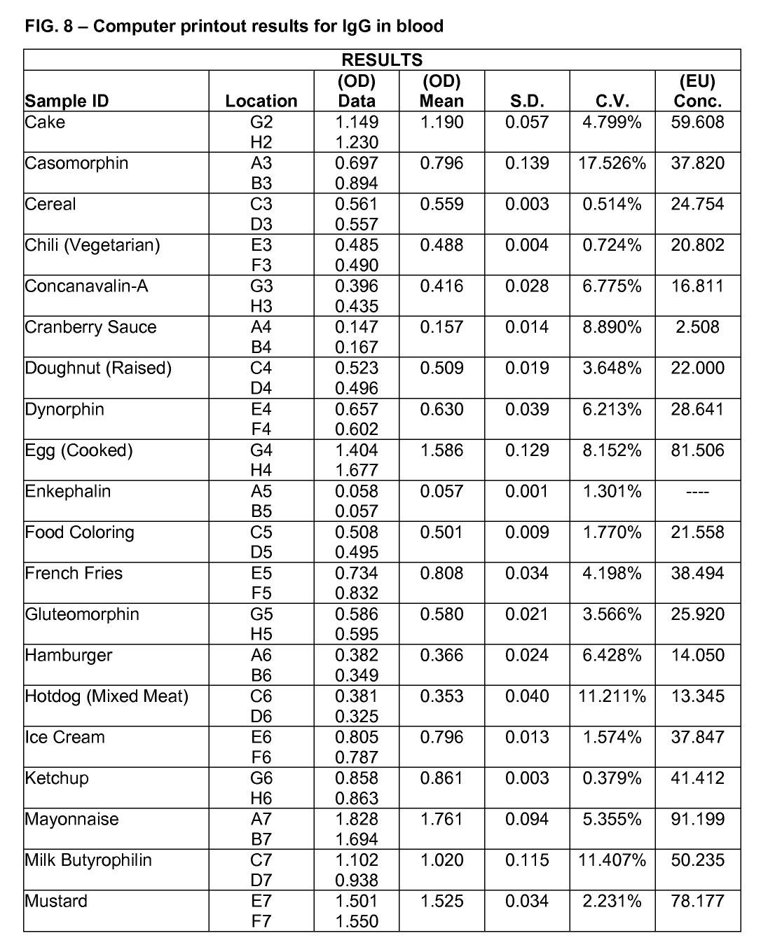

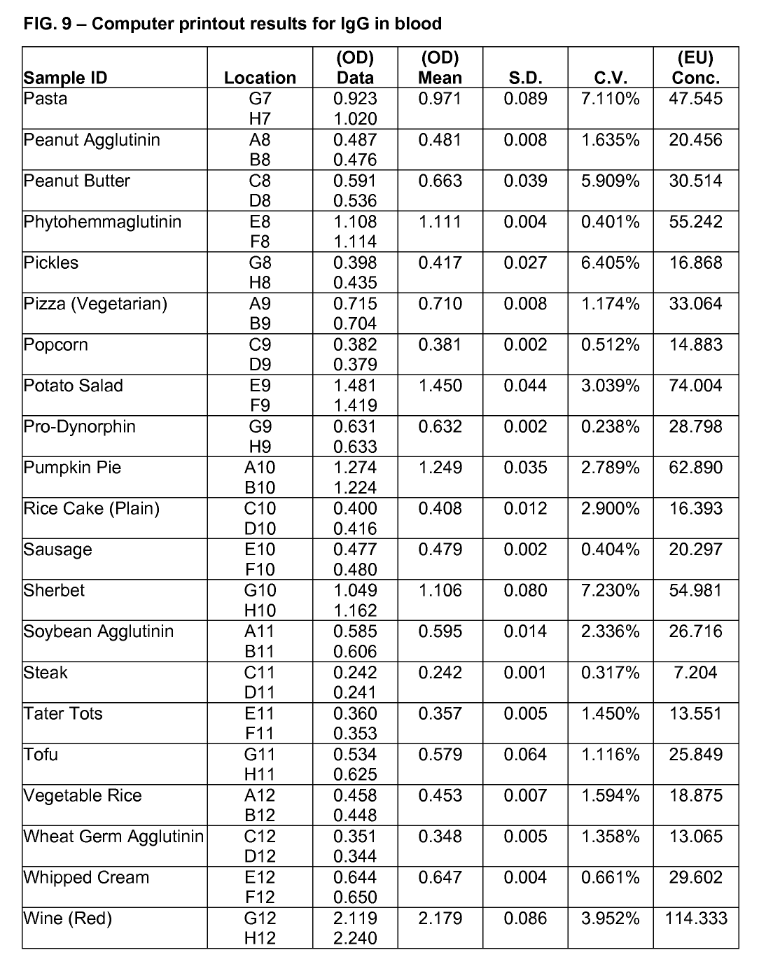

FIG. 6-9
Computer printout results for IgG in blood.

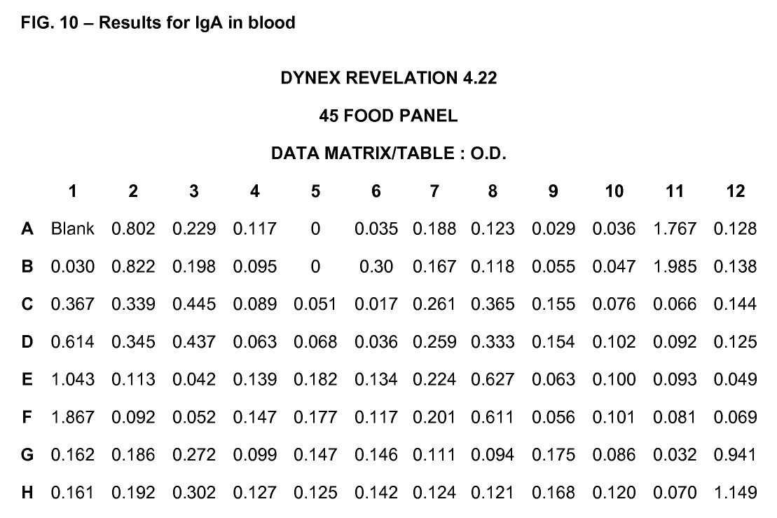

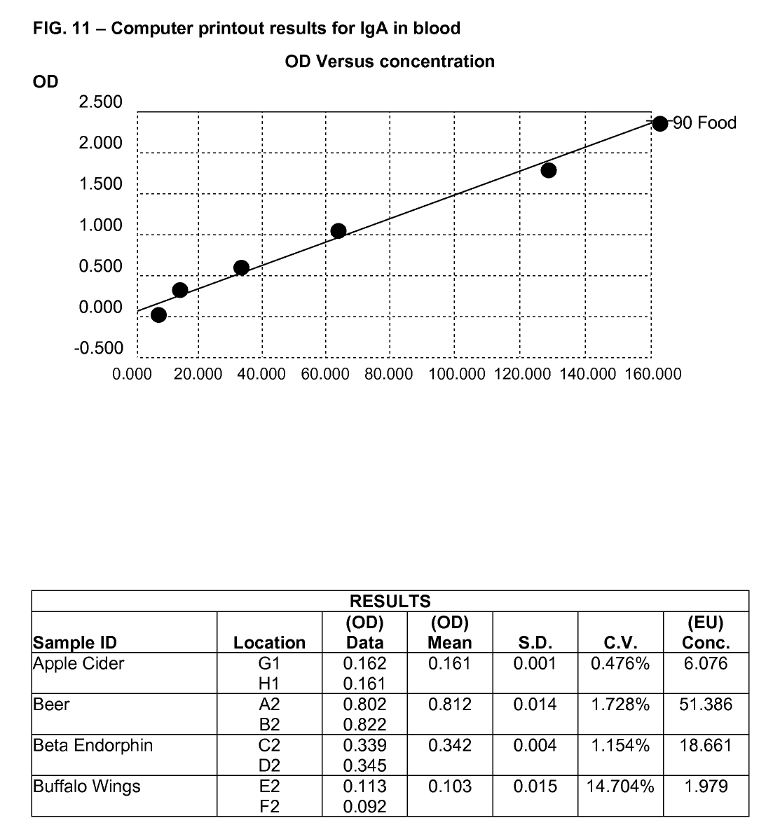

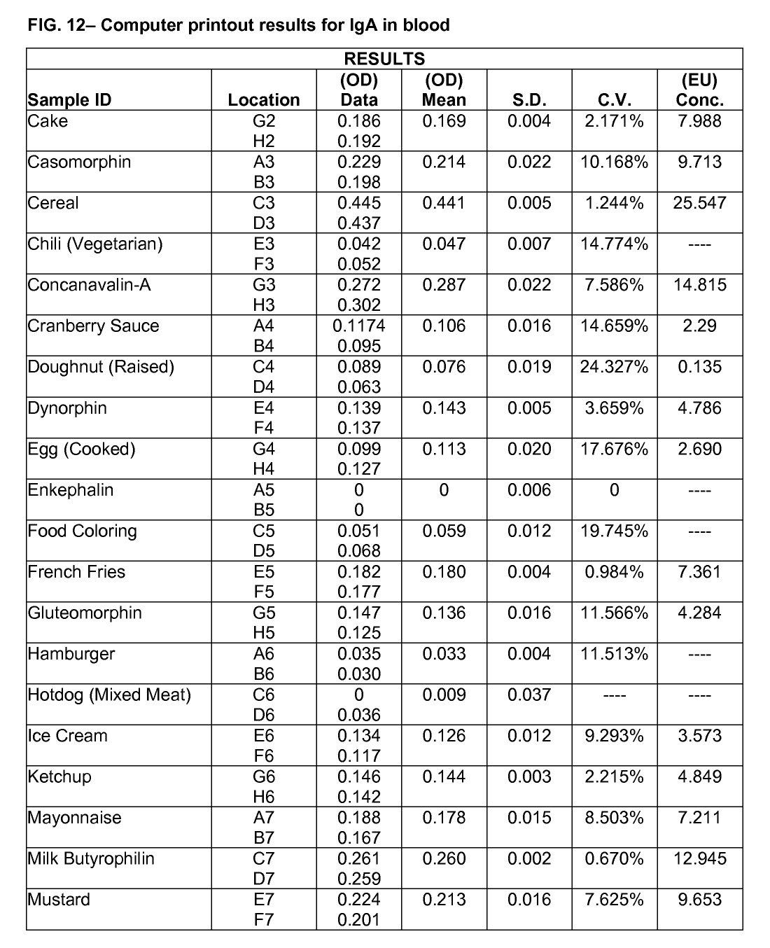

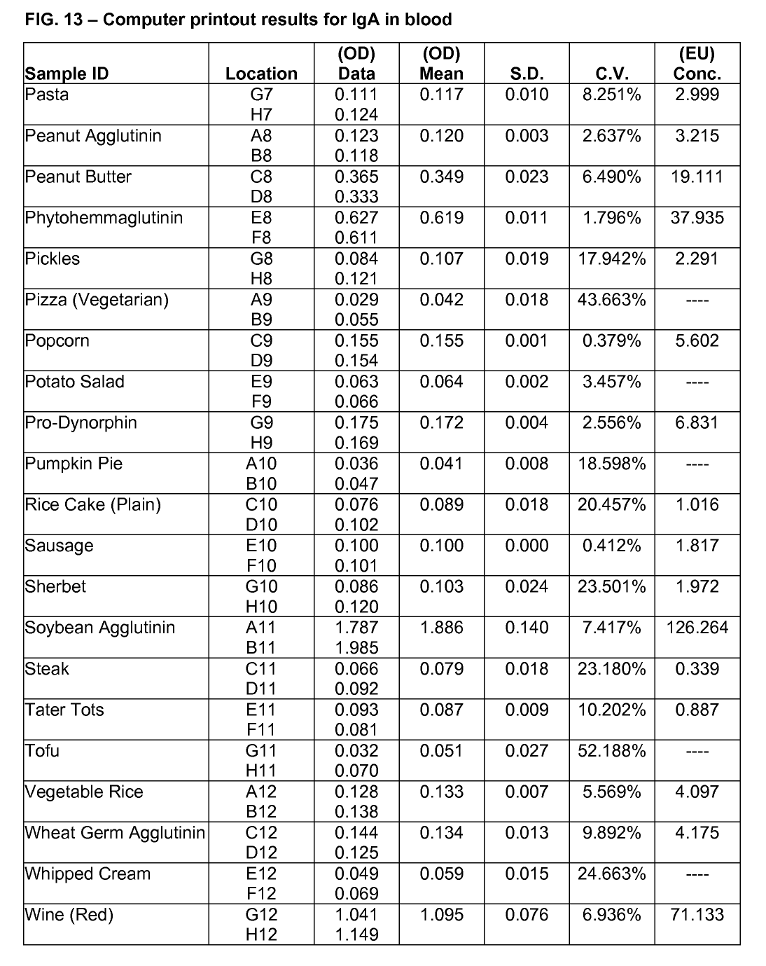

FIG. 10-13
Computer printout results for IgA in blood.

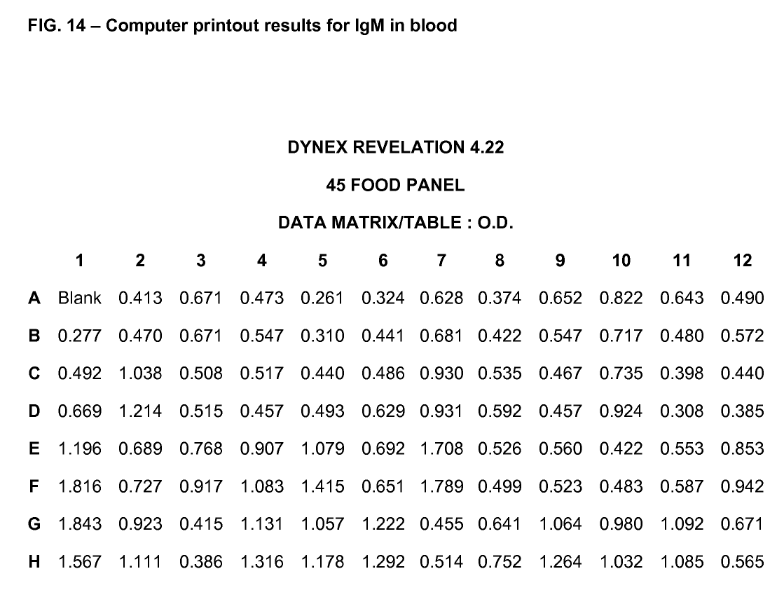

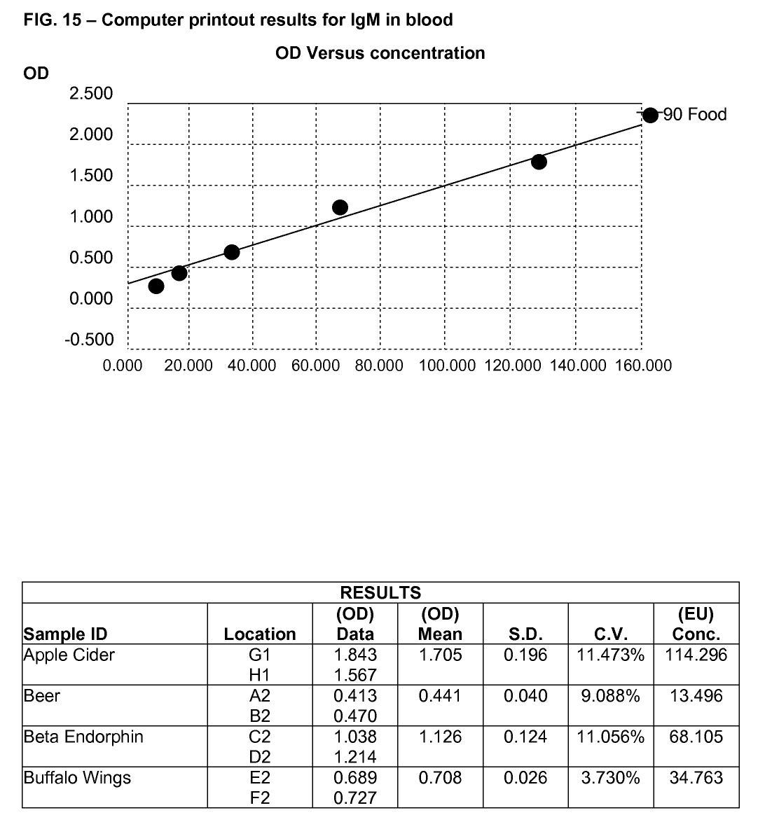

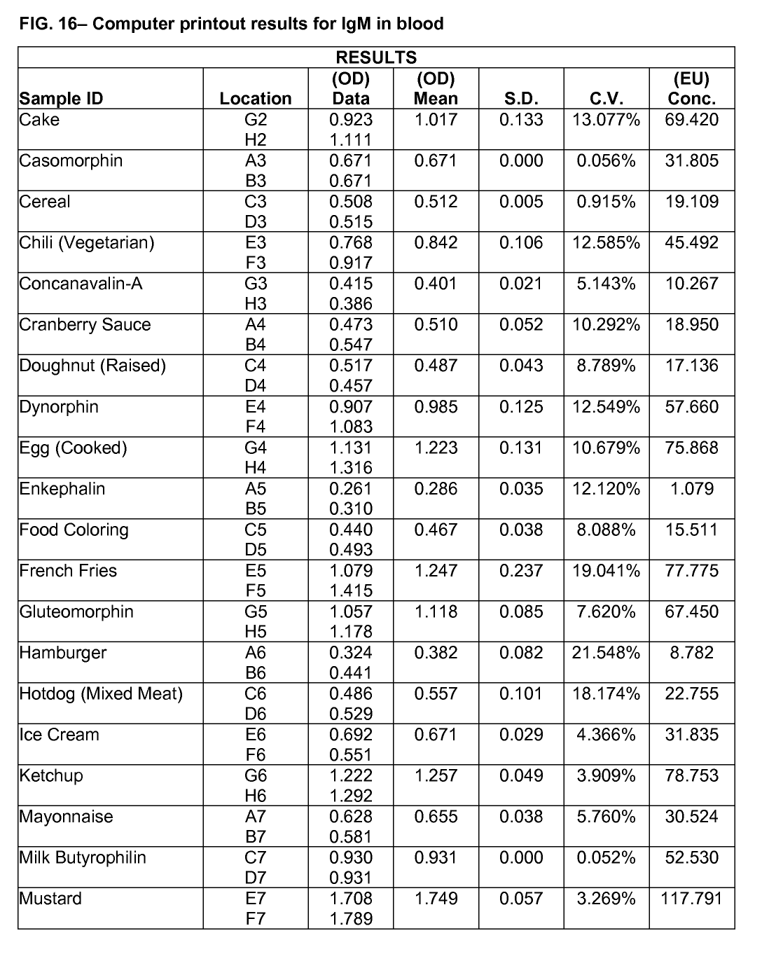

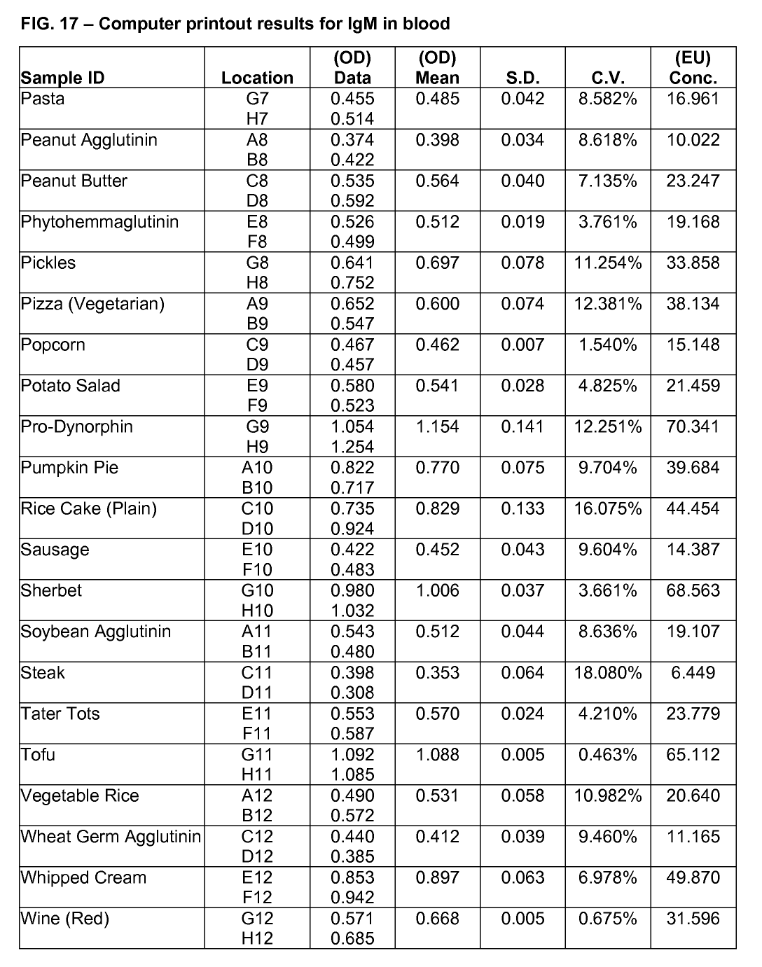

FIG. 14-17
Computer printout results for IgM in blood.

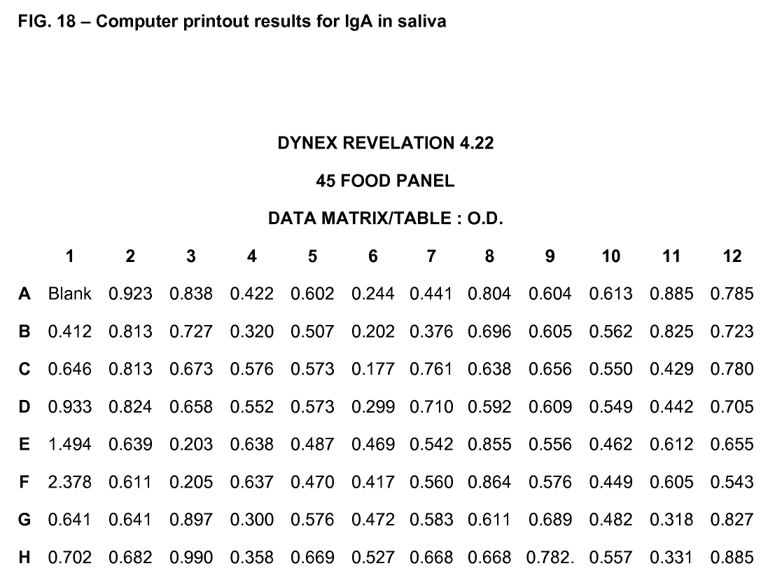

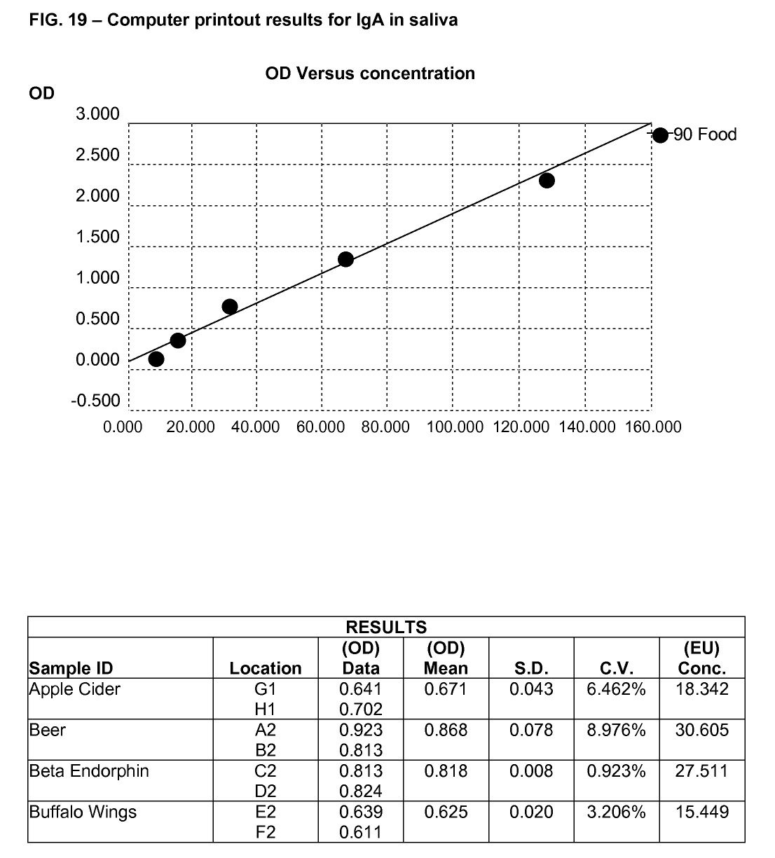

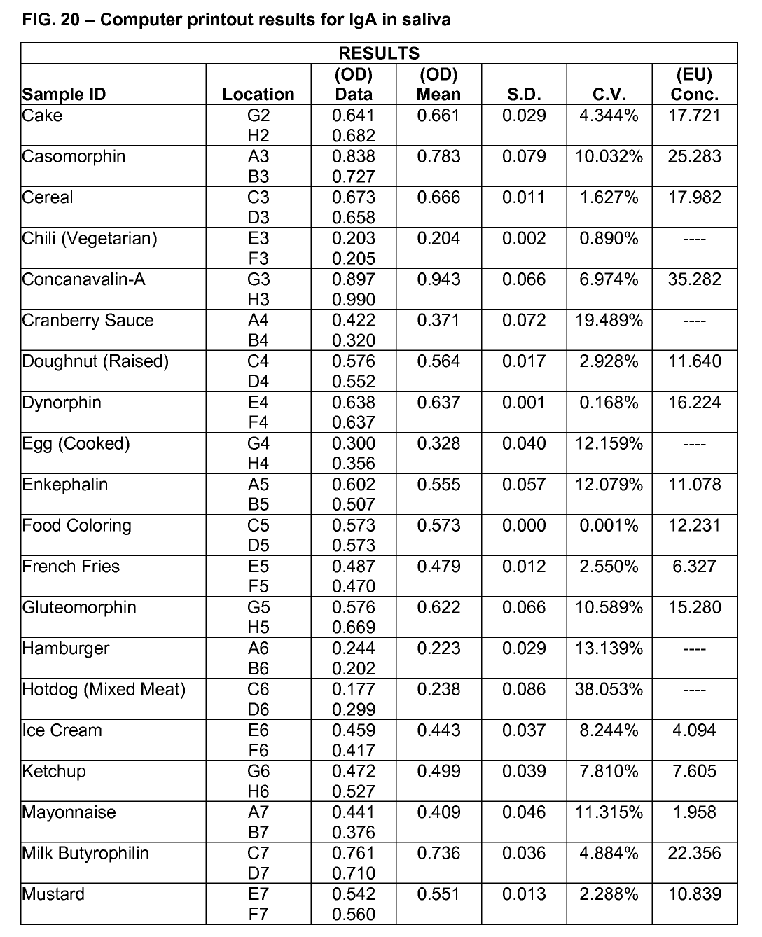

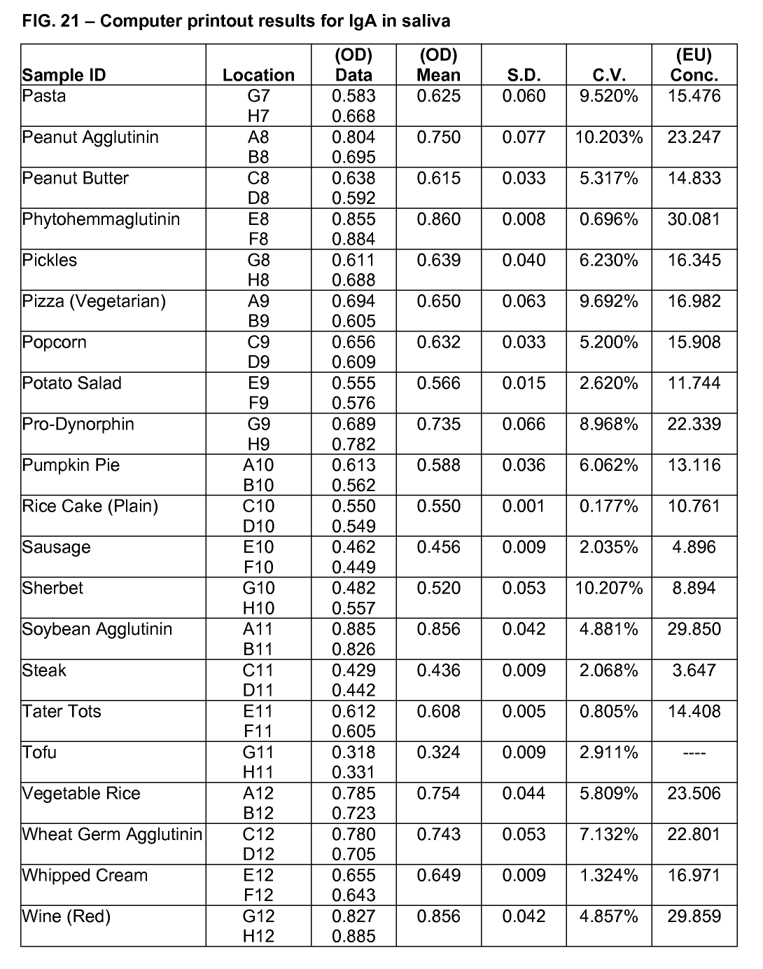

FIG. 18-21
Computer printout results for IgA+IgM in saliva.

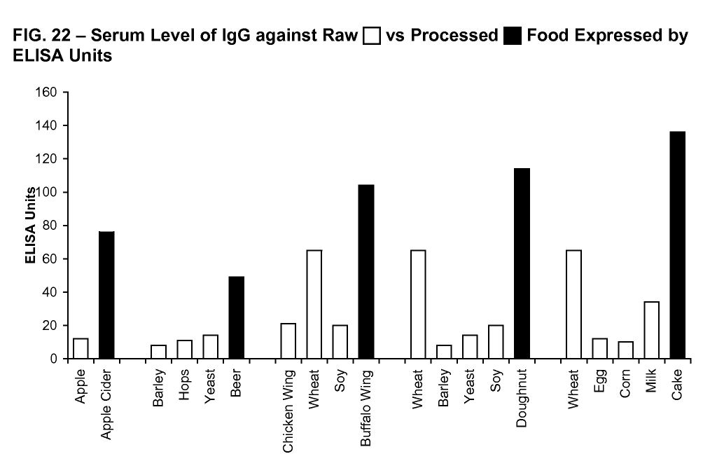

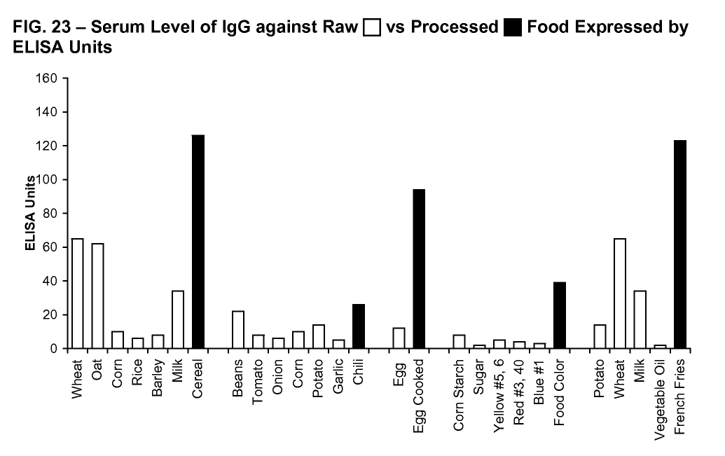

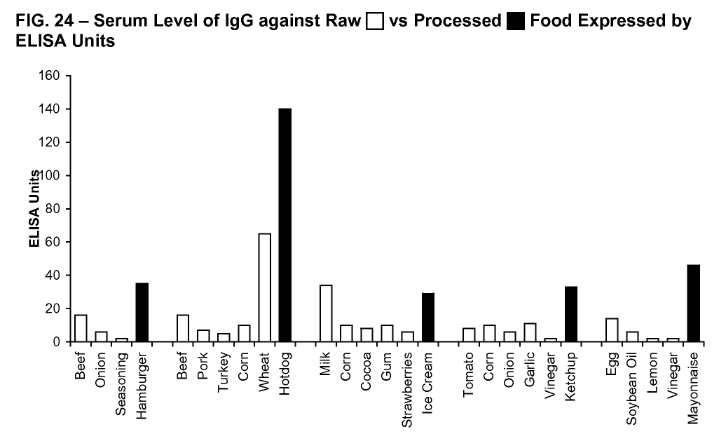

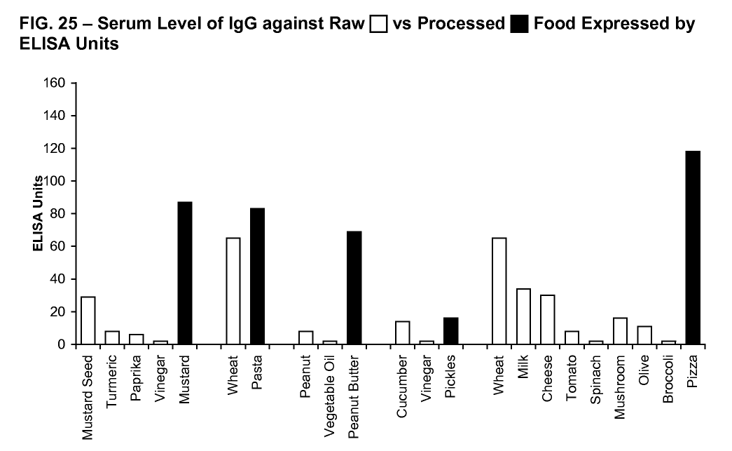

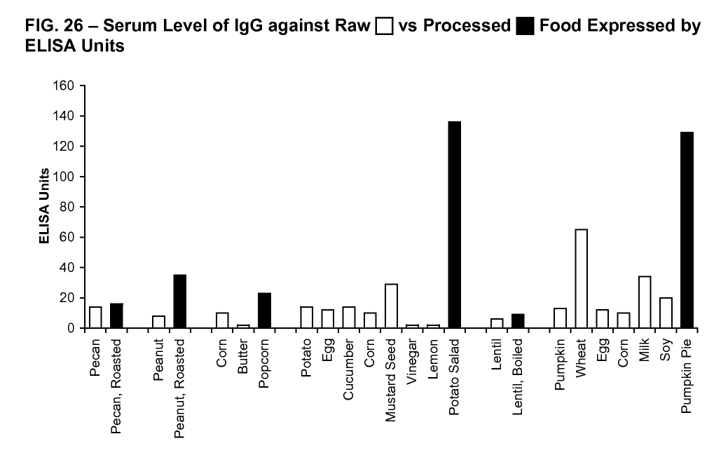

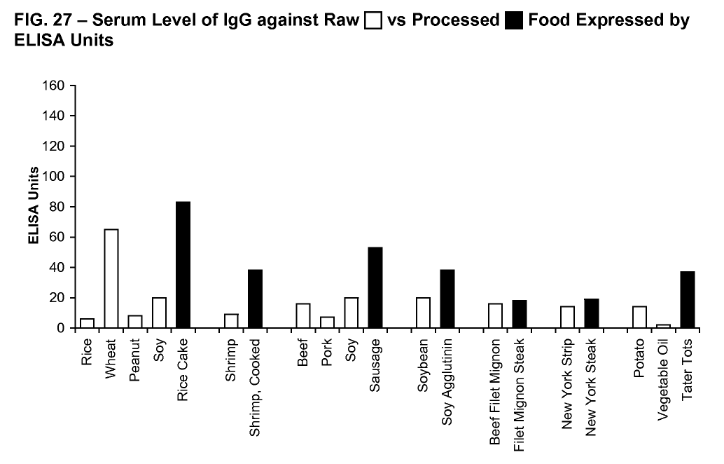

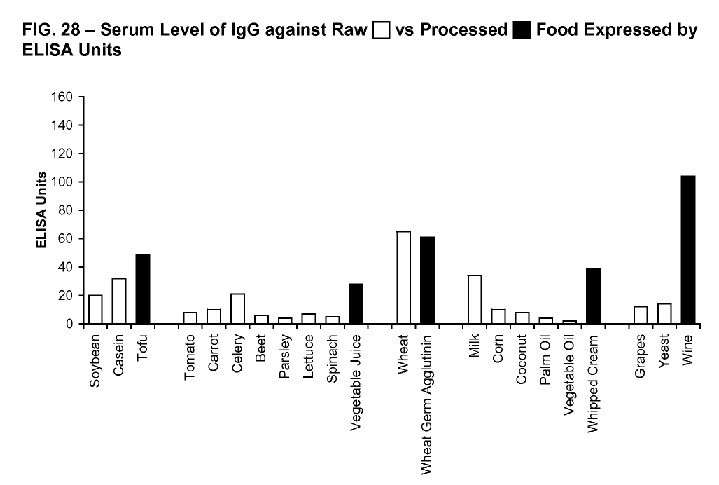

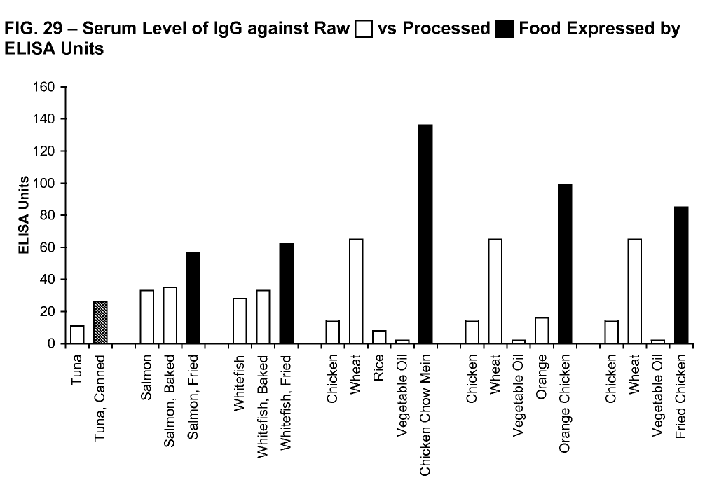

FIG. 22-29
Serum level of IgG against raw vs processed food expressed in ELISA units.

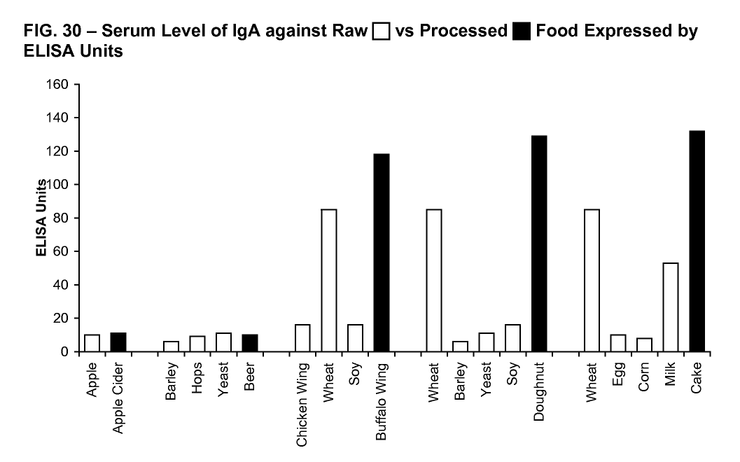

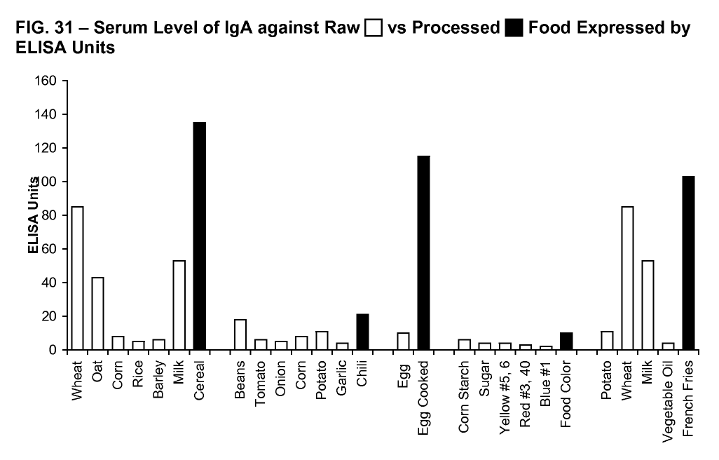

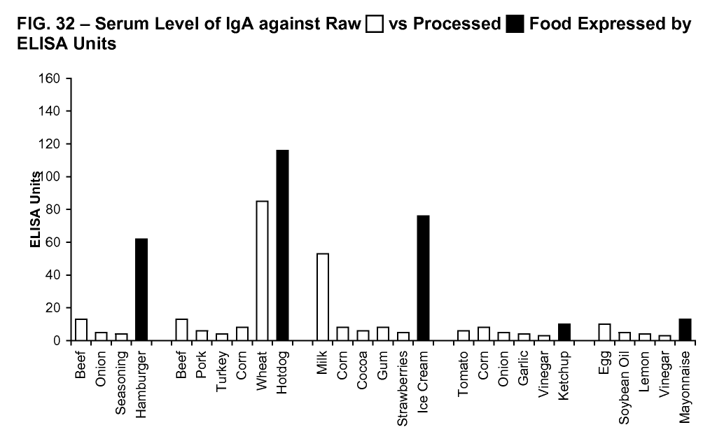

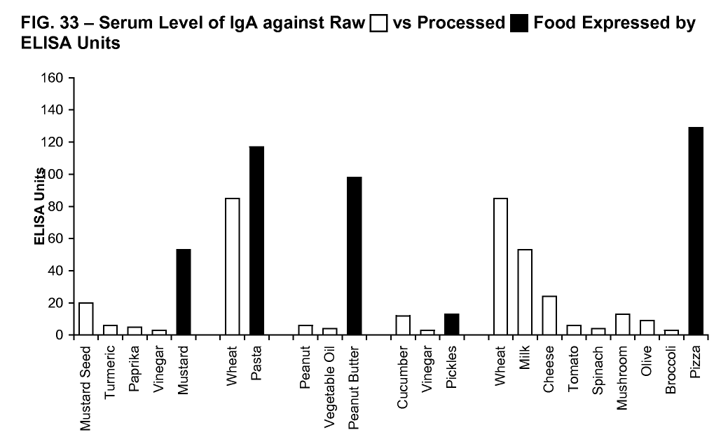

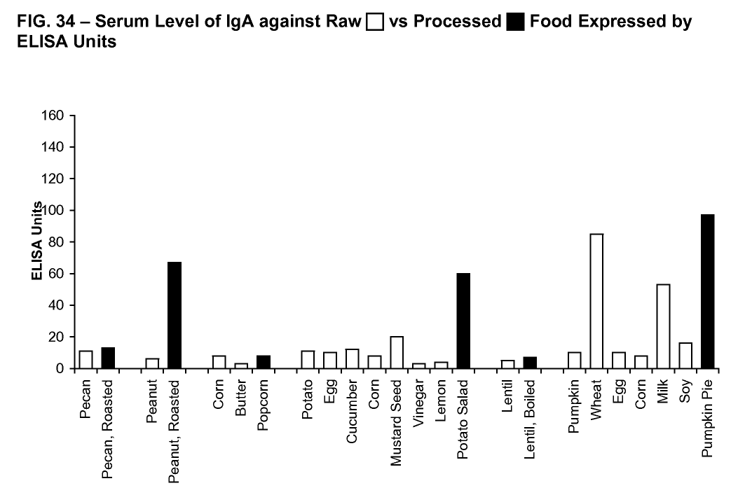

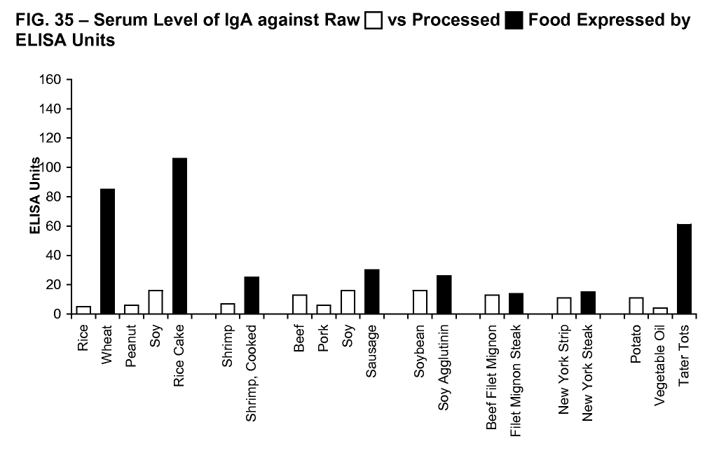

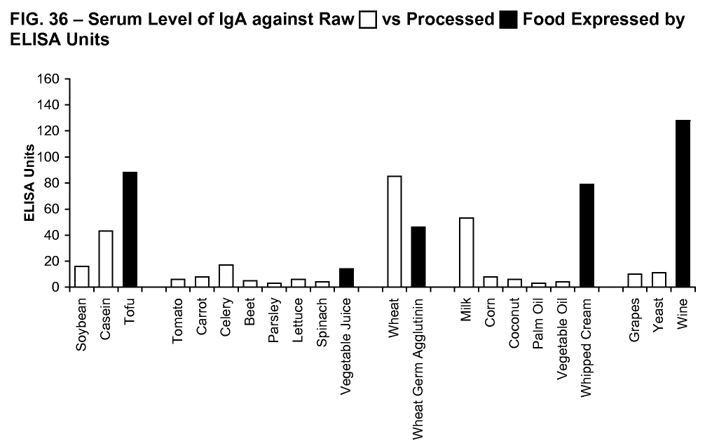

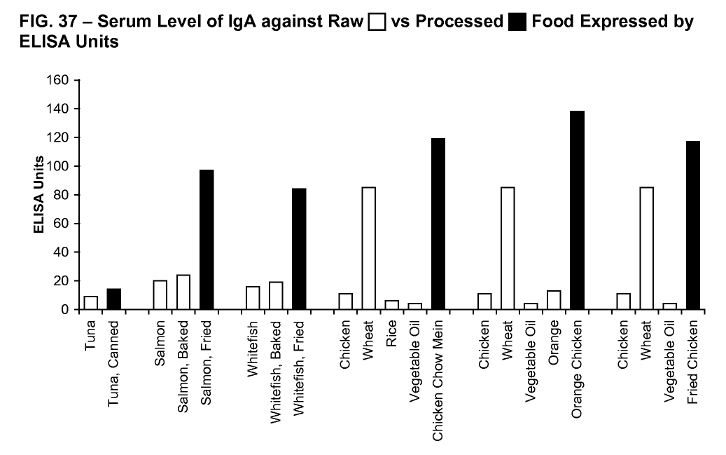

FIG. 30-37
Serum level of IgA against raw vs processed food expressed by ELISA units.

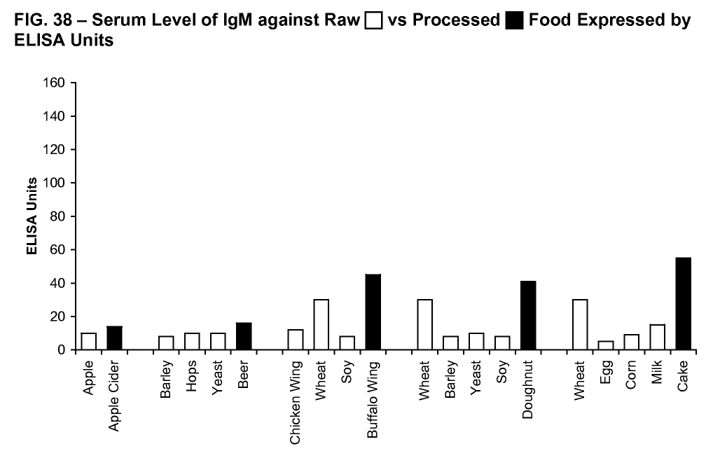

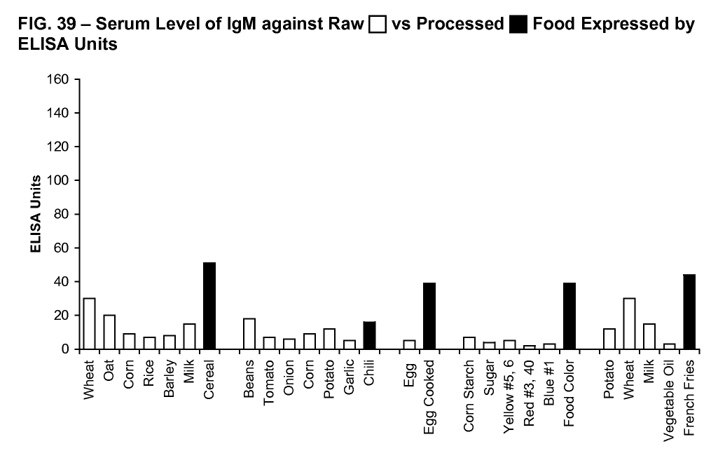

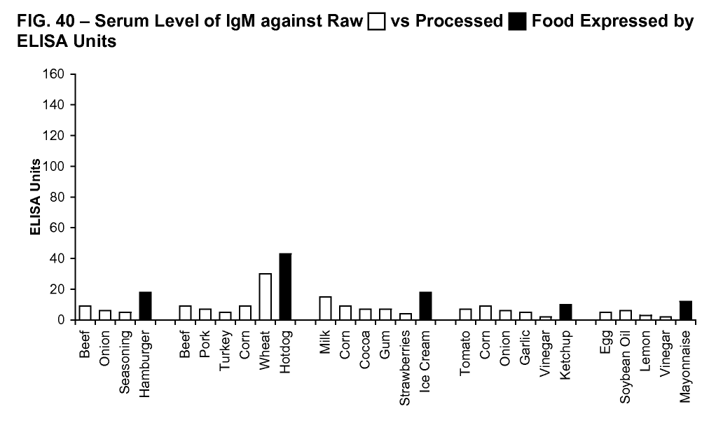

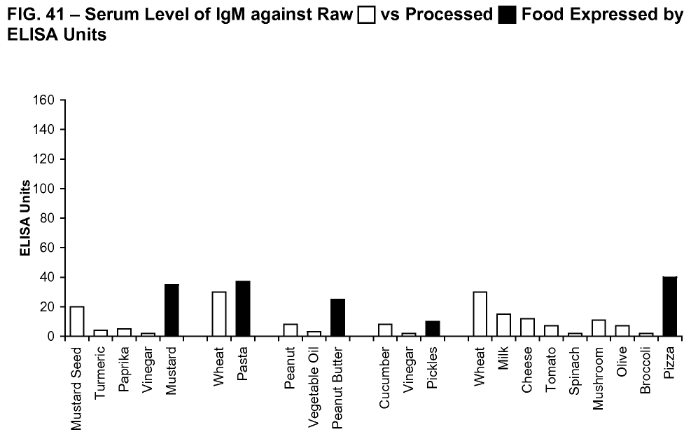

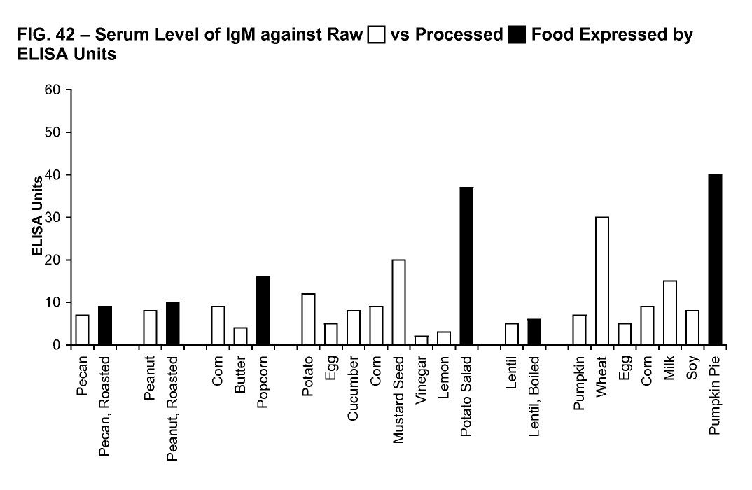

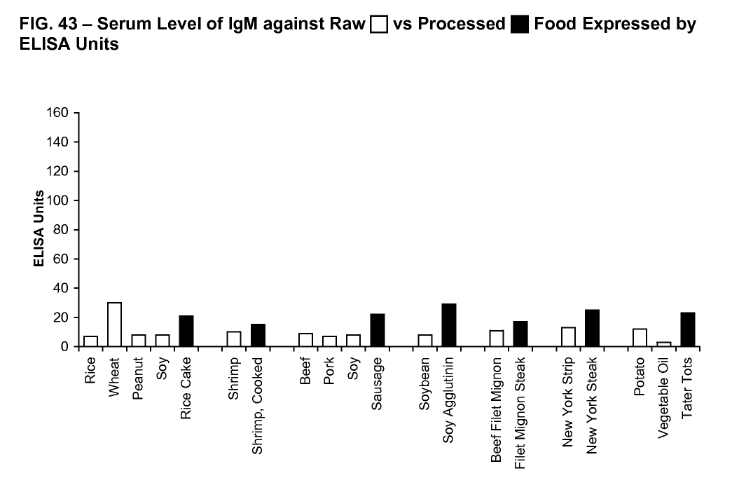

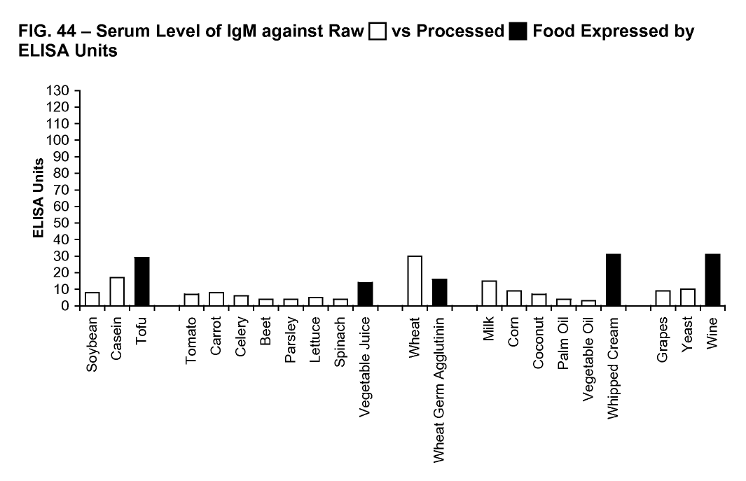

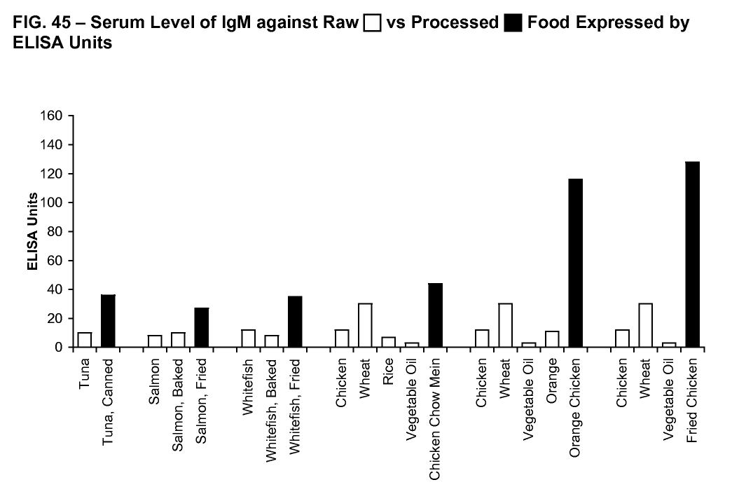

FIG. 38-45
Serum level of IgM against raw vs processed food expressed by ELISA units.

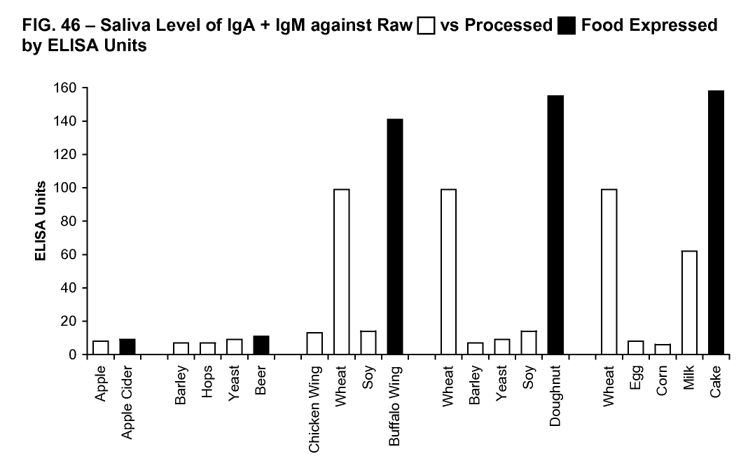

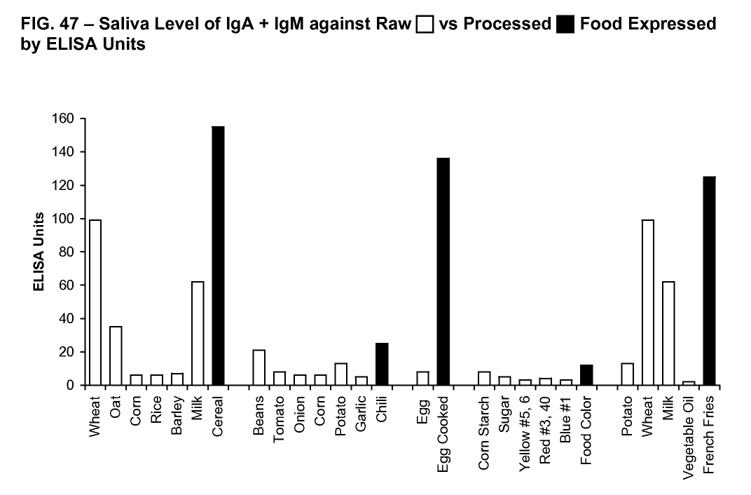

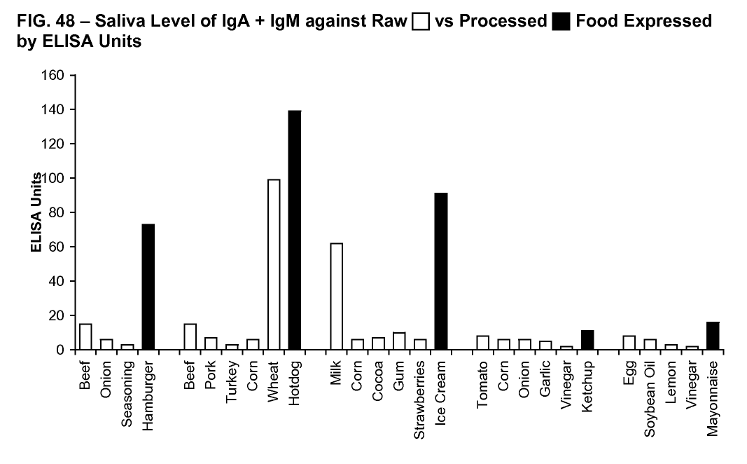

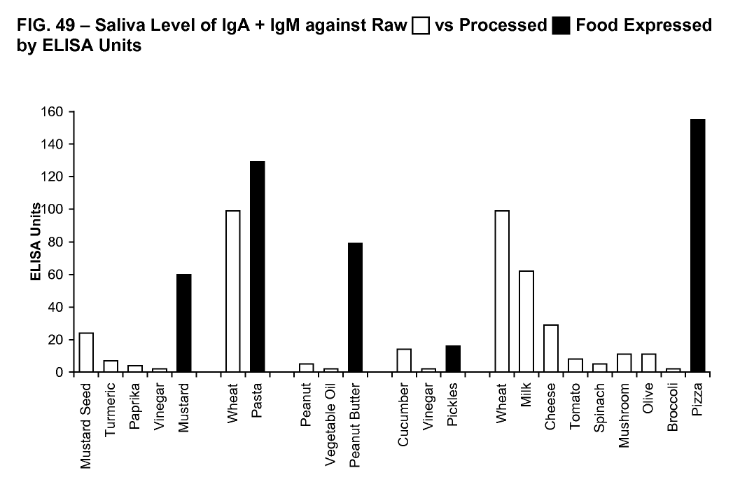

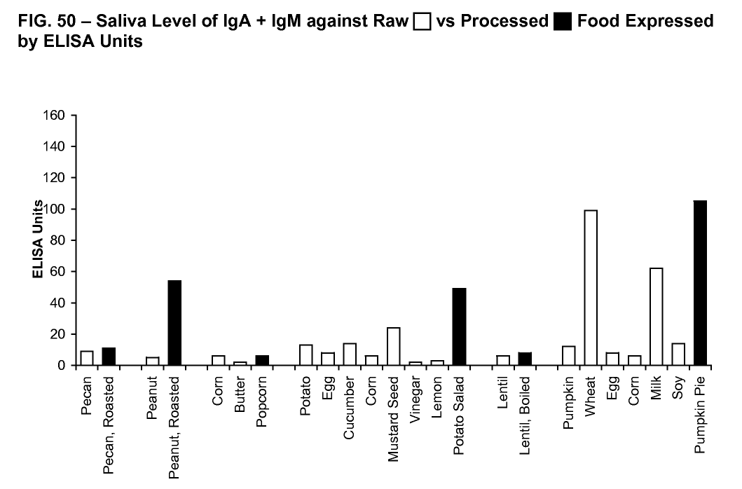

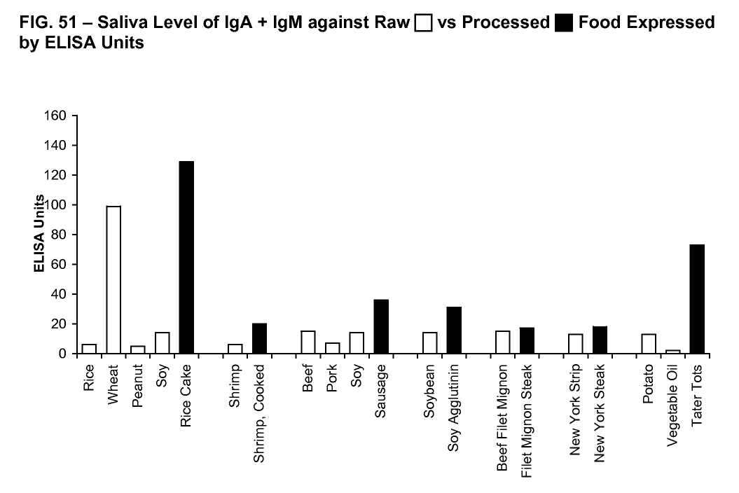

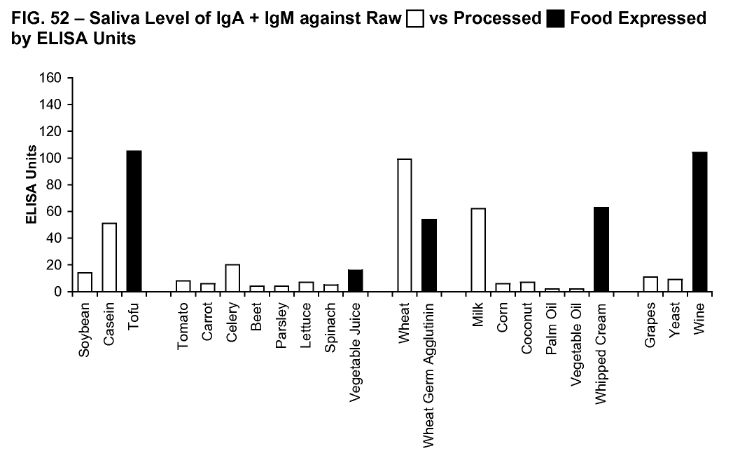

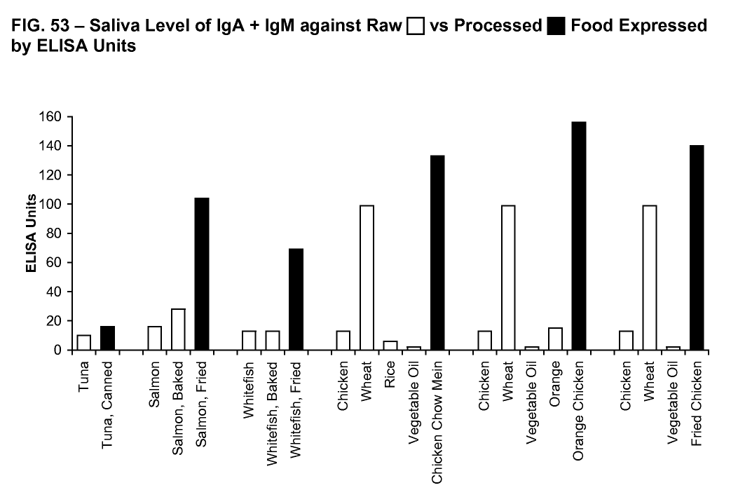

FIG. 46-53
Saliva level of IgA+IgM against raw vs processed food expressed by ELISA units.

### DETAILED DESCRIPTION OF THE PREFERRED EMBODIMENTS

We have developed a blood and saliva test that will accurately inform a physician of clinical conditions used to diagnose patients who may suffer from delayed food allergies or food intolerance to modified foods. The test utilizes a method that measures antibody titers to modified dietary antigens. The test can also utilize a test method that measures the antibodies' ability to bind to peptides prepared by enzymatic digestion corresponding to the dietary antigen. In order to assist the physician to make a more etiologic based diagnosis, we have developed an immunoassay for detecting food allergies and food intolerance in a patient using blood and saliva.

The test involves using a method for detecting food allergies and food intolerance in a patient. The method includes (a) determining a level of antibodies against a modified dietary food antigen in blood or saliva from the patient; and (b) comparing the level determined in step (a) with normal levels of the antibodies in said blood and saliva samples.

The possible outcomes for the comparison include (i) lower than normal or about normal levels of antibodies to modified dietary food antigens indicate optimal conditions; and (ii) higher than normal levels of antibodies to modified dietary food antigens indicate a food allergy or food intolerance.

Delayed food sensitivity is a growing concern for practitioners in many medical fields as it is associated with a multitude of disorders, such as multiple sclerosis, autism and rheumatoid arthritis, and affects an estimated 40% of the population. Patients presenting with clustered symptoms of migraine, mood swings, fatigue, intestinal upset, joint pain, high blood pressure and attention problems are often found to have delayed immune reaction to food antigens. Thus, it is vital to offer the medical community scientifically supported and sensitive food intolerance testing.

One reason traditional food sensitivity testing fails is that it does not reflect a true, real-world, contemporary, non-raw food diet. Widely-used food antigen testing uses only raw food isolates. Since the discovery of fire, fewer and fewer people consume a diet consisting solely of raw foods. As was shown in the previous sections, researchers have demonstrated that chemical/molecular changes occur during food preparation, cooking and processing. In addition to altering the makeup of a single food, other changes to the food can occur when this food is combined with another during cooking and processing. Thus, a person who is allergic to ketchup may not have an immune reaction to a raw tomato. If tested using traditional food sensitivity assays, this patient would result negative for tomato, and the patient's problems would be unresolved.

Armed with the desire to perfect delayed food sensitivity testing, these assays were designed for those consuming a contemporary American diet. Products were purchased at large supermarket chains to reflect American purchasing and accessibility of food products. Each advertised ingredient of the prepared, cooked and processed foods was carefully catalogued at the lab. Some examples of the raw and processed foods used for the extraction of antigens are shown in Table 2.

Dietary antigens of the preferred embodiments are classified into the general groups as followed: milk and milk products; eggs and egg products; meat and meat products; fish, mollusks, and crustaceans and their products; oils, fats, and their products; grains and grain products; pulses, seeds, kernels, nuts, and their products; vegetables and vegetable products; fruits and fruit products; sugar, sugar products, chocolate products, and confectionery; and spices and herbs.

“Milk and milk products” include, but are not limited to, American cheese, cheddar cheese, cottage cheese, cow's milk, goat's milk, Swiss cheese, and yoghurt.

“Eggs and egg products” include, but is not limited to, eggs.

“Meat and meat products” include, but are not limited to, beef, chicken, pork, and turkey.

“Fish, mollusks, and crustaceans and their products” include, but are not limited to, clam, codfish, crab, halibut, lobster, oyster, salmon, sardine, scallop, shrimp, sole, trout, and tuna.

“Oils, fats, and their products” include, but is not limited to, butter, vegetable oil, and soybean oil.

“Grains and grain products” include, but are not limited to, barley, buckwheat, malt, oat, rice, rye, and wheat.

“Pulses, seeds, kernels, nuts, and their products” include, but are not limited to, almond, cashew, coffee, cola nut, lima bean, millet, peanut, pinto bean, safflower seed, sesame, soybean, sunflower seed, and walnut.

“Vegetables and vegetable products” include, but are not limited to, broccoli, cabbage, carrot, cauliflower, celery, corn, cucumber, eggplant, green pea, green pepper, iceberg lettuce, mushroom, onion, potato, spinach, squash, string bean, sweet potato, and tomato.

“Fruits and fruit products” include, but are not limited to, apple, avocado, banana, blueberry, cantaloupe, grape, grapefruit, lemon, olive, orange, peach, pineapple, and strawberry.

“Sugar, sugar products, chocolate products, and confectionery” include, but are not limited to, chocolate, honey, and cane sugar.

“Spices and herbs” include, but are not limited to, chili powder, cinnamon, garlic, mustard seed, parsley, tea, and yeast.

### Example 1

### Analytical Methods for Identification and Characterization of Modified Food Antigens or Allergens

The isolation of proteins and glycoproteins is a prerequisite for extraction of antigens deom modified foodstuffs. Each raw or processed food was ground at 4° C. using a food processor and extraction buffers and reagents, such as Coco buffer (0.55% NaHCO₃, 1% NaCl), 0.1M phosphate buffer saline pH 7.4, 70% ethanol, and cold acetone.

Each food was mixed in different buffers and kept on the stirrer for 2 h at room temperature. After centrifugation at 2000 g for 15 minutes the liquid phase containing proteins, glycoproteins and lipoproteins were removed and dialysed against 0.01M PBS using dialysis bags with a cutoff of 6,000. Dialysis was repeated for three times in order to make sure that all non-antigenic materials are removed. After dialysis extracted antigens from the above conditions were combined, and protein concentrations were measured.

The separation of the different proteins from food is carried out by applying chromatographic and electrophoretic methods. The electrophoretic methods include sodium dodecylsulfate polyacrylamide gel electrophoresis (SDS-PAGE) and isoelectric focusing (IEF). In the case of SDS-PAGE, the separation of the proteins is carried out according to their molecular weight. On the other hand, IEF is used to separate proteins and peptides by their isoelectric points. The electrophoretically-separated proteins in polyacrylamide gels are visualized by silver staining or Coomassie brilliant blue staining.

Apart from electrophoretic techniques, immunological methods are used with regard to the identification and characterization of allergens. The specific determination of food allergens can be carried out by immunoblotting and enzyme linked immunosorbent assays (ELISA). FIG. 4 shows a diagram of a representative procedure for preparation of dietary antigens and peptides used in our U.S. Pat. No. 6,689,569.
Under these controlled conditions the exact amount of raw or processed food antigens used for coating microplates is the same in each preparation. For each antigen this coating was done in duplicate. Binding each of these food antigens to the wells of the microtiter plates in duplicate not only increases the reproducibility of the IgG, IgM and IgA assays but results in greater specificity and sensitivity, producing a better clinical outcome. For instance, testing a patient against raw food alone, such as raw egg, may not show a delayed food allergic reaction, but testing the same patient against antigens prepared from cooked egg might result in a severe immune reaction.

### Example 2

### Assay Procedure for Detection of IgA and IgM in Saliva and IgG, IgM or IgA in Blood Against Raw and Processed Food Antigens

Dietary proteins and peptides were dissolved in phosphate buffered saline (PBS) at a concentration of 1.0 mg/ml then diluted 1:100 in different buffers, including 0.1 M carbonate-bicarbonate buffer, pH 9.5, or phosphate buffer saline, pH 7.4. 50 μl or more were added to each well of a polystyrene flat-bottom ELISA plate. Plates were incubated overnight at 4° C. and then washed three times with 20 mM Tris-buffered saline (TBS) containing 0.05% Tween 20. After washing, the plates were coated with 1.5% BSA and 1.5% gelatin in TBS and then incubated for 2 hours at room temperature and then overnight at 4° C. After the overnight incubation, the BSA+gelatin was removed. Plates were washed three times with 20 mM Tris-buffered saline (TBS) containing 0.05% Tween 20, dried and stored at 4° C.

Quality control was performed by the addition of serum or saliva with low, medium and high titers of antibodies. In addition, plates were studied for the detection of non-specific reaction to the microwell plates. Without the addition of serum or saliva, the plates underwent the complete ELISA procedure to verify that there was no non-specific binding. After the performance of Quality Control, the plates were kept at 4° C. until used.

### Example 3

### Test for Antibodies to Dietary Antigens

The immunoassay can use patient saliva or blood collected in a sterile tube. Serum and saliva can be stored frozen for up to six months at −40° C.

The purified antigens were prepared according to Example 1 and were immobilized by attachment to a solid surface, such as a microtiter plate. Defined above, the dietary antigens are derived from the following general groups: milk and milk products; eggs and egg products; meat and meat products; fish, mollusks, and crustaceans and their products; oils, fats, and their products; grains and grain products; pulses, seeds, kernels, nuts, and their products; vegetables and vegetable products; fruits and fruit products; sugar, sugar products, chocolate products, and confectionery; and spices and herbs. These foods in their raw or processed form are listed in Table #2.

Test tubes, microtiter plates, nitrocellulose paper or other matrices were coated with about 1-10 micrograms of food antigens in 0.1 M PBS pH 7.4 or in carbonate buffer pH 9.5. The pH of the buffers used for coating may vary from as low as 6.0 to as high as a pH of 10.

Sera samples were collected by venipuncture and allowed to sit for 20 min at room temperature. After centrifugation for 10 min at 800 g the serum was removed and stored at −40° C.

Saliva samples were collected using sterile tubes. The samples were collected in the morning, before brushing teeth, smoking or drinking, and then every 4 hours until midnight. About 5 mL of saliva was collected. After a gentle chewing action to stimulate saliva production, the sample was collected in a test tube containing about 0.1 mL of preservative. Stored saliva was frozen at −40° C. or lower in tightly sealed sterile tubes. Samples were not repeatedly frozen and thawed and were not stored in self-defrosting freezers because the sample would desiccate and/or immunoglobulin degradation would occur.

The wash buffer was made as follows: in a 500 mL graduated cylinder, 450 mL of water was added to 50 mL of 10× wash buffer. The solution was mixed and transferred to a 500 mL squeeze bottle and stored at 2-8° C. until used. Then, 20 mL of conjugate diluent was added to the anti-human IgG, IgA or IgM conjugate and mixed well.

The substrate buffer and stop solution were ready for use. (CAUTION: Both solutions are caustic: avoid contact with skin and eyes; rinse with copious amounts of water in the event of contact.)

The substrate solution was prepared only immediately before use. For 1-5 strips, 5 mL of substrate buffer were pipetted into the empty substrate reconstitution bottle and 1 substrate tablet was dropped in. The bottle was shaken to dissolve the tablet. The buffer was used within an hour after reconstitution as recommended.

TABLE #2
Examples of raw and processed foods used for antigenic extraction
and measurement of IgG, IgM and IgA antibodies in blood and saliva

| A - Raw Food | | B- Processed Food |
|---|---|---|
| 1. | Apple | Apple Cider |
| 2. | Pork | Bacon |
| 3. | Barley, Hops, Yeast | Beer |
| 4. | Chicken Wing, Wheat, | Buffalo Wing |
| | Soy | |
| 5. | Wheat, Barley, Yeast, | Doughnut |
| | Soy, Sugar | |
| 6. | Wheat, Egg, Corn, | Cake |
| | Milk, Sugar | |
| 7. | Wheat, Oat, Corn, | Cereal |
| | Rice, Barley, Milk, | |
| | Sugar | |
| 8. | Chicken, Wheat, Rice, | Chicken Chow Mein |
| | Veg. Oil | |
| 9. | Chicken, Wheat, Veg. | Chicken, Fried |
| | Oil | |
| 10. | Chicken, Wheat, | Chicken, Orange |
| | Orange, Veg. Oil | |
| 11. | Beans, Chili Powder, | Chili (Vegetarian) |
| | Tomato, Onion, Corn, | |
| | Potato, Garlic | |
| 12. | Coffee | Coffee, Roasted |
| 13. | Cranberry, Corn | Cranberry Sauce |
| 14. | Egg, Raw | Egg, Cooked |
| 15. | Egg, Assorted Vegs., | Egg Roll |
| | Wheat, Soy | |
| 16. | Corn Starch, Sugar | Food Coloring |
| | Dextrose, Yellow #5, | |
| | Yellow #6, Red #3, | |
| | Red #40, Blue #1 | |
| 17. | Potato, Veg. Oil, | French Fries |
| | Wheat, Milk | |
| 18. | Beef, Onion, | Hamburger |
| | Seasoning | |
| 19. | Beef, Pork, Turkey, | Hotdog |
| | Corn, Wheat | |
| 20. | Milk, Corn, Cocoa, | Ice Cream |
| | Gum, Strawberries, | |
| | Sugar | |
| 21. | Tomato, Corn, Onion, | Ketchup |
| | Garlic, Vinegar | |
| 22. | Lentil | Lentil, Boiled |
| 23. | Egg, Soybean Oil, | Mayonnaise |
| | Lemon, Vinegar | |
| 24. | Mustard Seed, | Mustard |
| | Turmeric, Paprika, | |
| | Vinegar | |
| 25. | Peanut, Veg. Oil | Peanut Butter |
| 26. | Wheat (Semolina) | Pasta |
| 27. | Peanut | Peanut, Roasted |
| 28. | Pecan | Pecan, Roasted |
| 29. | Cucumber, Vinegar | Pickles |
| 30. | Wheat, Milk, Cheese, | Pizza |
| | Olives, Tomato, | |
| | Spinach, Mushroom, | |
| | Broccoli | |
| 31. | Corn, Butter | Popcorn |
| 32. | Egg, Potato, | Potato Salad |
| | Cucumber, Corn, | |
| | Mustard Seed, | |
| | Vinegar, Lemon | |
| 33. | Pumpkin, Wheat, Egg, | Pumpkin Pie |
| | Corn, Milk, Soy | |
| 34. | Rice, Wheat, Peanut, | Rice Cake |
| | Soy | |
| 35. | Salmon | Salmon, Baked |
| 36. | Salmon | Salmon, Fried |
| 37. | Beef, Pork, Soy | Sausage |
| 38. | Shrimp | Shrimp, Cooked |
| 39. | Soybean | Soy Agglutinin |
| 40. | Beef Filet Mignon | Steak, Filet Mignon |
| 41. | New York Strip | Steak, New York |
| 42. | Potato, Veg. Oil | Tater Tots |
| 43. | Soybean, Casein | Tofu |
| 44. | Tuna | Tuna, Canned |
| 45. | Tomato, Carrot, | Vegetable Juice |
| | Celery, Beet, Parsley, | |
| | Lettuce, Spinach | |
| 46. | Wheat | Wheat Germ Agglutinin |
| 47. | Milk, Corn, Coconut, | Whipped Cream |
| | Palm Oil, Veg. Oil | |
| 48. | Whitefish | Whitefish, Baked |
| 49. | Whitefish | Whitefish, Fried |
| 50. | Grape, Yeast | Wine |

Serum and saliva were prepared as follows. All strips to be used, reagents, controls, and patient's serum and saliva were equilibrated to room temperature (22-25° C.). Patient's saliva was diluted 1:5 with saliva diluent buffer, 2 mL saliva+8 mL buffer. Saliva dilutions were made in tubes prior to addition to wells and thoroughly mixed before dispensing. Dilution of saliva could be as low as 1:2 or as high as 1:20. The patient's serum is diluted 1:200 by the addition of 2011 of serum to 4 mL of serum diluent buffer. This dilution of serum could be as low as 1:50 and as high as 1:600.

The use of two wells per test was necessary. Preferably, for every determination, at least 12 strips of eight wells were used to run blank calibrators and patient's samples.

Well identification: The antigen-coated strips were used. Each was divided into 8 equal-sized squares, as shown in FIG. 5. In strip #1, the top well is labeled “Blank” and the next wells are labeled Calibrator 1, Calibrator 2, Calibrator 3, Calibrator 4, and Calibrator 5. The last two wells and the other eleven strips are labeled foods #1-45. For additional food testing more 8×12 strip plates may be used.

The assay procedure was as follows: 100 μl of specimen diluent buffer was pipetted to two blank wells. 100 mL each of Calibrators 1-5 was then pipetted into identified wells, after which the patient's samples were added to duplicate wells. The reagents were dispensed slowly to avoid splashing and air bubbles. If large air bubbles occurred, they were aspirated or the plate was gently shaken. The plate was covered and incubated for 60 min at room temperature (about 22-25° C.). Specimen was shaken from the wells into a container containing disinfectant solution or aspirated with a vacuum device. All wells were emptied prior to filling with 1× wash buffer and allowing a 10-20 sec soak time. The wells were emptied by shaking into a disposal container or aspirated. Washing was repeated three more times. The inverted plate was tapped onto a paper towel to completely remove all residual liquid. Then, 100 μl of anti-IgA, IgM or IgG antibody labeled with enzyme was added to the tested plates. The plate was covered and incubated for 60 min at room temperature (22-25° C.). The liquid was shaken or aspirated from all the wells and washed four times. Then 100 μl of p-NPP substrate was added to all the wells at timed intervals that corresponded to the reading time of the instrument used to read the reactions. The 45-60 min incubation time was started as substrate was added to the first well. The plate was covered and incubated 45-60 min at 22-25° C. (the assay may be incubated for less than 45 min if incubation temperature is higher than 25° C.). Then, 50 μl of 3N NaOH was pipetted into all the wells at the same timed intervals that the p-NPP was added. The plate was shaken for 1-2 min by hand or shaker, avoiding splashing. The bottom of the wells was wiped with a non-abrasive paper towel and the instrument was zeroed on the blank well. The O.D. was read at 405±5 nm within 30 min and reactions recorded.

The units or titer of IgA, IgM or IgG antibody against specific foods were determined by a computer program and the use of the following formula:

$$\begin{matrix}Titer of IgA , IgM \\ Or IgG Antibody in Blood \\ Or Saliva ( Concentration Values )\end{matrix} = \frac{Values of calibrators \times Abosrbance of test specimen}{Absorbance of calibrators}$$

For precise determination, absorbances were converted to concentration values using a point-to-point data reduction method. (However, one may substitute a best-fit linear regression program to obtain values). If a program is used to provide calculation of concentration values, the calibrator concentration values (which appear on vial label) should be entered as the “standards.”

The values were obtained manually and plotted using linear graph paper. The X-axis was each calibrator's concentration value. The Y-axis was the corresponding mean absorbance value. A best-fit line was drawn. The concentration of each patient's saliva or serum was obtained by locating its absorbance on the Y-axis and finding the corresponding concentration value on the X-axis.

### Example 4

### Analysis of Results

Results were analyzed as a panel. The dietary antigens were selected from cheese, eggs, beef, chicken, pork, turkey, whitefish, salmon, shrimp, tuna, butter, vegetable oil, soybean oil, barley, malt, oat, rice, wheat, coffee, peanut, pinto bean, sesame, soybean, broccoli, cabbage, carrot, celery, corn, cucumber, iceberg lettuce, lentils, mushroom, onion, potato, spinach, tomato, apple, grape, lemon, olive, orange, strawberry, cocoa, sugar, chili powder, garlic, mustard seed, parsley, and yeast.

Computer-generated printouts for IgG, IgM and IgA in serum as well as IgA+IgM in saliva against 45 different dietary proteins and peptides are shown in FIGS. 6-21. Calibration graphs were obtained from the optical density values resulting from the calibration samples for each run. Note that the optical densities are converted to ELISA units based on the titer of antibodies from five different calibrators ranging from 8-128 ELISA units. The calibration graphs can be used to extrapolate concentration values from optical density values obtained from the testing discussed in Example 3. The concentration values of IgG, IgA or IgM are compiled for the set of dietary antigens for each healthy control and patient.

FIGS. 6-9 show calibration graphs of optical density for binding reactions versus concentration of food IgG in serum. The concentration of antibodies expressed by ELISA units against 45 different foods is shown. Note in this example that the patient produced high IgG levels of antibodies against beer, mayonnaise, mustard, and wine.

FIGS. 10-13 show calibration graphs of optical density for binding reactions versus concentration of food IgA in serum. The concentration of antibodies expressed by ELISA units against 45 different foods is shown. Note in this example that the patient produced high IgA levels of antibodies against beer, soybean agglutinin and wine.

FIGS. 14-17 show calibration graphs of optical density for binding reactions versus concentration of food IgM in serum. The concentration of antibodies expressed by ELISA units against 45 different foods is shown. Note in this example that the patient produced high IgM levels of antibodies against apple cider, cooked egg, french fries, ketchup, mustard, sherbet and tofu.

FIGS. 18-21 show calibration graphs of optical density for binding reactions versus concentration of food IgA+IgM in saliva. The concentration of antibodies expressed by ELISA units against 45 different foods is shown. Note in this example that patient produced high IgA levels of antibodies in saliva against beer and wine.

Diagrams of patients with normal or abnormal levels of IgG, IgM, and IgA in blood and saliva against raw and processed foods are shown in FIGS. 22-53. Note that many antibody reactions are enhanced when compared to their content of raw food antigens.

FIGS. 22-29 show the concentration of IgG antibodies in serum expressed by ELISA units against raw versus processed foods. Note in this example the high levels expressed against a majority of the processed or modified versions of the foods in comparison to their raw or crude forms: whereas raw apple has a value of 12 EU, apple cider has a value of 76 EU; barley, hops and yeast are 8, 11, and 14 EU, respectively, but beer is 49 EU; chicken wing, wheat and soy are 21, 65 and 20, but buffalo wing is 104; wheat, barley, yeast and soy are 65, 8, 14 and 20, but doughnut is 114; wheat, egg, corn and milk are 65, 12, 10 and 34, but cake is 136; wheat, oat, corn, rice, barley and milk are 65, 62, 10, 6, 8 and 34, but cereal is 126; raw egg is 12, but cooked egg is 94; corn starch, sugar, Yellow #5 and #6, Red #3 and #40, and Blue #1 are 8, 2, 5, 4 and 3, but food coloring is 39; potato, wheat, milk and vegetable oil are 14, 65, 34 and 2, but french fries are 123; beef, onion and seasoning are 16, 6 and 2, but hamburger is 35; beef, pork, turkey, corn and wheat are 16, 7, 5, 10 and 65, but hotdog is 140; tomato, corn, onion, garlic and vinegar are 8, 10, 6, 11 and 2, but ketchup is 33; egg, soybean oil, lemon and vinegar are 14, 6, 2 and 2, but mayonnaise is 46; mustard seed, turmeric, paprika and vinegar are 29, 8, 6 and 2, but mustard is 87; raw wheat is 65, but pasta is 83; peanut and vegetable oil are 8 and 2, but peanut butter is 69; wheat, milk, cheese, tomato, spinach, mushroom olive and broccoli are 65, 34, 30, 8, 2, 16, 11 and 2, but pizza is 118; raw peanut is 8, but roasted peanut is 35; corn and butter are 10 and 2, but popcorn is 23; potato, egg, cucumber, corn, mustard seed, vinegar and lemon are 14, 12, 14, 10, 29, 2 and 2, but potato salad is 136; pumpkin, wheat, egg, corn, milk and soy are 13, 65, 12, 10, 34 and 20, but pumpkin pie is 129; rice, wheat, peanut and soy are 6, 65, 8 and 20, but rice cake is 83; raw shrimp is 9, but cooked shrimp is 38; beef, pork and soy are 16, 7 and 20, but sausage is 53; soybean is 20, but soybean agglutinin is 38; potato and vegetable oil are 14 and 2, but tater tots are 37; soybean and casein are 20 and 32, but tofu is 49; grapes and yeast are 12 and 14, but wine is 104; tuna is 11, but canned tuna is 26; raw salmon is 33, but fried salmon is 57; raw whitefish is 28, but fried whitefish is 62; chicken, wheat, rice and vegetable oil are 14, 65, 8 and 2, but chicken chow mein is 136; chicken, wheat, vegetable oil and orange are 14, 65, 2 and 16, but orange chicken is 99; chicken, wheat and vegetable oil are 14, 65 and 2, but fried chicken is 85.

FIGS. 30-37 show the concentration of IgA antibodies in serum expressed by ELISA units against raw versus processed foods. Note in this example the high levels expressed against a majority of the processed or modified versions of the foods in comparison to their raw or crude forms: whereas chicken wing, wheat and soy have values of 21, 65 and 20 EU respectively, buffalo wing is 118; wheat, barley, yeast and soy are 85, 6, 11 and 16, but doughnut is 129; wheat, egg, corn and milk are 85, 10, 8 and 53, but cake is 132; wheat, oat, corn, rice, barley and milk are 85, 43, 8, 5, 6 and 53, but cereal is 135; raw egg is 10, but cooked egg is 115; potato, wheat, milk and vegetable oil are 11, 85, 53 and 4, but french fries are 103; beef, onion and seasoning are 13, 5 and 4, but hamburger is 62; beef, pork, turkey, corn and wheat are 13, 6, 4, 8 and 85, but hotdog is 116; milk, corn, cocoa, gum and strawberries are 53, 8, 6, 8 and 5, but ice cream is 76; mustard seed, turmeric, paprika and vinegar are 20, 6, 5 and 3, but mustard is 53; raw wheat is 85, but pasta is 117; peanut and vegetable oil are 6 and 4, but peanut butter is 98; wheat, milk, cheese, tomato, spinach, mushroom olive and broccoli are 85, 53, 24, 6, 4, 13, 9 and 3, but pizza is 129; raw peanut is 6, but roasted peanut is 67; potato, egg, cucumber, corn, mustard seed, vinegar and lemon are 11, 10, 12, 8, 20, 3 and 5, but potato salad is 60; pumpkin, wheat, egg, corn, milk and soy are 10, 85, 10, 8, 53 and 16, but pumpkin pie is 97; rice, wheat, peanut and soy are 5, 85, 6 and 16, but rice cake is 106; raw shrimp is 7, but cooked shrimp is 25; beef, pork and soy are 13, 6 and 16, but sausage is 30; soybean is 16, but soybean agglutinin is 26; potato and vegetable oil are 11 and 4, but tater tots are 61; soybean and casein are 16 and 43, but tofu is 88; grapes and yeast are 10 and 11, but wine is 128; raw salmon is 20, but fried salmon is 97; raw whitefish is 16, but fried whitefish is 84; chicken, wheat, rice and vegetable oil are 11, 85, 6 and 4, but chicken chow mein is 119; chicken, wheat, vegetable oil and orange are 11, 85, 4 and 13, but orange chicken is 138; chicken, wheat and vegetable oil are 11, 85 and 4, but fried chicken is 117.

FIGS. 38-45 show the concentration of IgM antibodies in serum expressed by ELISA units against raw versus processed foods. Note in this example the high levels expressed against a majority of the processed or modified versions of the foods in comparison to their raw or crude forms: whereas raw apple has a value of 10 EU, apple cider has a value of 14 EU; barley, hops and yeast are 8, 10, and 10 EU, respectively, but beer is 16 EU; chicken wing, wheat and soy are 12, 30 and 8, but buffalo wing is 45; wheat, barley, yeast and soy are 30, 8, 10 and 8, but doughnut is 41; wheat, egg, corn and milk are 30, 5, 9 and 15, but cake is 55; wheat, oat, corn, rice, barley and milk are 30, 20, 9, 7, 8 and 15, but cereal is 51; raw egg is 5, but cooked egg is 39; corn starch, sugar, Yellow #5 and #6, Red #3 and #40, and Blue #1 are 7, 4, 5, 2 and 3, but food coloring is 39; potato, wheat, milk and vegetable oil are 12, 30, 15 and 3, but french fries are 44; beef, onion and seasoning are 9, 6 and 5, but hamburger is 18; beef, pork, turkey, corn and wheat are 9, 7, 5, 9 and 30, but hotdog is 43; egg, soybean oil, lemon and vinegar are 5, 6, 3 and 2, but mayonnaise is 12; mustard seed, turmeric, paprika and vinegar are 20, 4, 5 and 2, but mustard is 35; raw wheat is 30, but pasta is 37; peanut and vegetable oil are 8 and 3, but peanut butter is 25; wheat, milk, cheese, tomato, spinach, mushroom olive and broccoli are 30, 15, 12, 7, 2, 11, 7 and 2, but pizza is 40; raw pecan is 7, but roasted pecan is 9; raw peanut is 8, but roasted peanut is 10; corn and butter are 9 and 4, but popcorn is 16; potato, egg, cucumber, corn, mustard seed, vinegar and lemon are 12, 5, 8, 9, 20, 2 and 3, but potato salad is 37; pumpkin, wheat, egg, corn, milk and soy are 7, 30, 5, 9, 15 and 8, but pumpkin pie is 40; raw shrimp is 10, but cooked shrimp is 15; beef, pork and soy are 9, 7 and 8, but sausage is 22; soybean is 8, but soybean agglutinin is 29; filet mignon beef is 11, but a filet mignon steak is 17; New York strip beef is 13, but a New York steak is 25; potato and vegetable oil are 12 and 3, but tater tots are 23; soybean and casein are 8 and 17, but tofu is 29; milk, corn, coconut, palm oil and vegetable oil are 15, 9, 7, 4 and 3, but whipped cream is 31; grapes and yeast are 9 and 10, but wine is 31; tuna is 10, but canned tuna is 36: raw salmon is 8, but fried salmon is 27; raw whitefish is 12, but fried whitefish is 35; chicken, wheat, rice and vegetable oil are 12, 30, 7 and 3, but chicken chow mein is 44; chicken, wheat, vegetable oil and orange are 12, 30, 3 and 11, but orange chicken is 116; chicken, wheat and vegetable oil are 12, 30 and 3, but fried chicken is 128.

FIGS. 46-53 show the concentration of IgA+IgM antibodies in saliva expressed by ELISA units against raw versus processed foods. Note in this example the high levels expressed against a majority of the processed or modified versions of the foods in comparison to their raw or crude forms: whereas chicken wing, wheat and soy have values of 13, 99 and 14 EU respectively, buffalo wing is 141; wheat, barley, yeast and soy are 99, 7, 9 and 14, but doughnut is 155; wheat, egg, corn and milk are 99, 8, 6 and 62, but cake is 158; wheat, oat, corn, rice, barley and milk are 99, 35, 6, 8, 7 and 62, but cereal is 155; raw egg is 8, but cooked egg is 136; potato, wheat, milk and vegetable oil are 13, 99, 62 and 2, but french fries are 125; beef, onion and seasoning are 15, 6 and 3, but hamburger is 73; beef, pork, turkey, corn and wheat are 15, 7, 3, 6 and 99, but hotdog is 139; milk, corn, cocoa, gum and strawberries are 62, 6, 7, 10 and 6, but ice cream is 91; egg, soybean oil, lemon and vinegar are 8, 6, 3 and 2, but mayonnaise is 16; mustard seed, turmeric, paprika and vinegar are 24, 7, 4 and 2, but mustard is 60; raw wheat is 99, but pasta is 129; peanut and vegetable oil are 5 and 2, but peanut butter is 79; wheat, milk, cheese, tomato, spinach, mushroom olive and broccoli are 99, 62, 29, 8, 5, 11, 11 and 2, but pizza is 155; raw peanut is 5, but roasted peanut is 54; potato, egg, cucumber, corn, mustard seed, vinegar and lemon are 13, 8, 14, 6, 24, 2 and 3, but potato salad is 49; pumpkin, wheat, egg, corn, milk and soy are 12, 99, 8, 6, 62 and 14, but pumpkin pie is 105; rice, wheat, peanut and soy are 6, 99, 5 and 14, but rice cake is 129; raw shrimp is 6, but cooked shrimp is 20; beef, pork and soy are 15, 7 and 14, but sausage is 36; soybean is 14, but soybean agglutinin is 31; potato and vegetable oil are 13 and 2, but tater tots are 73; soybean and casein are 14 and 51, but tofu is 105; grapes and yeast are 11 and 9, but wine is 104; raw salmon is 16, but fried salmon is 104; raw whitefish is 13, but fried whitefish is 69; chicken, wheat, rice and vegetable oil are 13, 99, 6 and 2, but chicken chow mein is 133; chicken, wheat, vegetable oil and orange are 13, 99, 2 and 15, but orange chicken is 156; chicken, wheat and vegetable oil are 13, 99 and 2, but fried chicken is 140.

### REFERENCES

Adams, D. H. and Eksteen, B. Aberrant homing of mucosal T cells and extra-intestinal manifestations of inflammatory bowel disease. Nat Rev Immunol, 2006, 6:244-251.
Al-Bayaty, H. F., et al. Salivary and serum antibodies to gliadin in the diagnosis of celiac disease. J Oral Pathol Med, 1989, 18: 578-581.
Barnes, R. M. R. IgG and IgA antibodies to dietary antigens in food allergy and intolerance. Clin Exp Allergy, 1995, 25:7-9.
Bengmark, S. Advanced glycation and lipoxidation end products—amplifiers of inflammation: the role of food. J Parenteral and Enteral Nutrition, 2007, 31:430-440.
Bischoff, S, and Crowe, S. E. Gastrointestinal food allergy: new insights into pathophysiology and clinical perspectives. Gastroenterology, 2005, 128:1089-1113.
Bock, A. U. and Atkins, F. M. Patterns of food hypersensitivity during sixteen years of double-blind placebo-controlled food challenges. J Pediatr, 1990, 117:561-567.
Brandtzaeg, P. Do salivary antibodies reliably reflect both mucosal and systemic immunity? Ann. N.Y. Acad. Sci. 2007, 1098: 288-311.
Brandtzaeg, P., et al, Human secretory immunoglobulins. I. Salivary secretions from individuals with normal or low levels of serum immunoglobulins. Scand J Haematol Suppl, 1970, 12: 1-83.
Brandtzeag, P. Role of secretory antibodies in the defense against infections. Int. J. Med. Microbiol, 2003, 293: 3-15.
Challacombe, S. J. The induction of secretory IgA responses in food allergy and intolerance edited by Brostoff J., Challacombe S. J., 1987, published by W.B. Sanders, Eastborne, England.
Chung, S. Y. and Champagne E. T. Association of end-product adducts with increased IgE binding of roasted peanuts. J Agric Food Chem, 2001, 49:3911-3916.
Codina, R., et al. Neoallergens in heated soybean hull. Int Arch Allergy Immunol, 1998, 117:120-125.
Czerinsky, C., et al. IgA antibody-producing cells in peripheral blood after antigen infestation: evidence for a common mucosal immune system in humans. Proc Natl Acad Sci, 1987, 84:2449-2453.
Davis, P. J. and Williams, S. C. Protein modification by thermal processing. Allergy, 1998, 53:102-105.
Doke, S., et al. Allergenicity of food proteins interacted with oxidized lipids in soybean-sensitive individuals. Agric Biol Chem, 1989, 53:1231-1235.
Eigenmann, P. A., et al. Prevalence of IgE-mediated food allergy among children with atopic dermatitis. Pediatrics, 1998, 101:E8.
Faria, A. M. and Weiner, H. L. Oral tolerance. Immunol Rev, 2005, 206:232-259.
Fasano, A. and Shea-Donohue, T. Mechanisms of disease: the role of intestinal barrier function in the pathogenesis of gastrointestinal autoimmune disease. Nat Clin Pract Gastroenterol Hepatol, 2005, 2:416-421.
Gahnberg, L. & b. Krasse, Salivary immunoglobulin A antibodies reacting with antigens from oral streptococci: longitudinal study in humans. Infect Immun, 1981, 33:697-703.
Gleeson, M., et al. Modifiers of the human mucosal immune system. Immunol Cell Biol, 1995, 73:397-404.
Gordon, B. R. Approaches to testing for food and chemical sensitivities. Otolaryngologic Clinics of North America, 2003, 36:917-940.
Hakeem, V., et al. Salivary IgA antigliadin antibody as a marker for celiac disease. Arch Dis Child, 1992, 67:724-727.
Hefle, S. L., et al. Soy sauce retains allergenicity through the fermentation production process. J Allergy Clin Immunol, 2005, 115:S32.
Hvatum, M., et al. Serum IgM subclass antibodies to a variety of food antigens in patients with celiac disease. Gut, 1992, 33:632-638.
Jertborn, M., et al. Saliva, breast milk, and serum antibody responses as indirect measures of intestinal immunity after oral cholera vaccination or natural disease. J Clin Microbiol, 1986, 24:203-209.
Kanda, M., et al. Detection and salivary increase of salivary antibodies to Staphylococcus lentus and indigenous bacteria in rabbit saliva, through a single tonsillar application of bacterial cells. Oral Microbiol Immunol, 2001, 16:257-264.
Kunisawa, J. and Kiyono, H. A marvel of mucosal T cells and secretory antibodies for the creation of first lines of defense. Cell Mol Life Sci, 2005, 62:1308-1321.
Leduc, V., et al. Anaphylaxis to wheat isolates: immunochemical study of a case proved by means of double-blind, placebo-controlled food challenge. J Allergy Clin Immunol, 2003, 111:897-899.
Malanin, K., et al. Anaphylactic reaction caused by neoallergens in heated pecan nut. Allergy, 1995, 50:988-991.
Maleki, S. J., et al. The effects of roasting on the allergenic properties of peanuts. J Allergy Clin Immunol, 2000, 106:763-768.
Nogueira, R. D., et al. Characterization of salivary immunoglobulin A responses in children heavily exposed to the oral bacterium Streptococcus mutans; influence of specific antigen recognition in infection. Infect Immun, 2005, 73:5675-5684.
Pecquet, C., et al. Is the application of cosmetics containing protein-derived products safe? Contact Dermatitis, 2002, 46:123.
Prausnitz, C. and Kustner, H. Studies on supersensitivity. Centralbl Bakteriol Abt Orig, 1921, 86:160-169.
Ramasamy, R., et al. Advanced glycation end products and RAGE: a common thread in aging diabetes, neurodegeneration, and inflammation. Glycobiology, 2005, 15:16R-28R.
Rosen, J., et al. Skin testing with natural foods in patients suspected of having food allergies: is it a necessity? J Allergy Clin Immunol, 1994, 1068-1070.
Rumbo, M., et al. Detection and characterization of antibodies specific to food antigens in human serum, saliva, colostrum and milk. Clin Exp Immunol, 1998, 112:453-458.
Sampson, H. A. Food allergy. Part I: immunopathogenesis and clinical disorders. J Allergy Clin Immunol, 1999, 103:717-728.
Sampson, H. A. Update on food allergy. J Allergy Clin Immunol, 2004, 113:805-819.
Sampson, H. A., et al. Fatal and near-fatal anaphylactic reactions to food in adolescents and children. N Engl J Med, 1992, 327:380-384.
Sanchez, C. and Fremont S. Consequences of heat treatment and processing of food on the structure and allergenicity of component proteins. Rev Fr Allergol Immunol Clin, 2003, 43:13-20.
Sandin, D. I., et al. Specific IgE determinations to crude and boiled lentil (Lens culinaris) extracts in lentil-sensitive children and controls. Allergy, 1999, 54(11): 1209-1214.
Sathe, S. K., et al. Effects of food processing on the stability of food allergens. Biotechnol Adv, 2005, 23:423-429.
Sen, M., et al. Protein structure plays a critical role in peanut allergen stability and may determine immunodominant IgE binding epitopes. J Immunol, 2002, 169:882-887.
Sicherer, S. H. and Sampson, H. A. Food allergy. J Allergy Clin Immunol, 2006, 117:S470-S475.
Spies, J. R. Allergens. J Agric Food Chem, 1974, 22(1):30-36.
Su, M., et al. Impact of γ-irradiation and thermal processing on the antigenicity of almond, cashew nut and walnut proteins. J Sci Food Agric, 2004, 84:1119-1125.
Taubman, M. A. and D. J. Smith. Significance of salivary antibody in dental disease. Ann Ny Y Acad Sci, 1993, 694: 202-215.
Walker, W. A. and Isselbacher, K. J. Intestinal antibodies. New Engl J Med, 1977, 297:767-773.

What is claimed is:

1- A method for determining the presence of delayed food allergy or food intolerance to processed or modified food antigens in a patient, comprising:
(a) Determining level of antibodies against modified or processed dietary food antigens present in blood and saliva samples from said patient.
(b) Antibodies are selected from the group consisting of IgG, IgM and IgA in blood and IgA, IgM in saliva.
(c) IgG, IgM and IgA in blood and IgA, IgM in saliva are antibody isotypes tested against said dietary protein-antigens or food isolates prepared from modified or processed food.
(d) Modification of food proteins or antigens is due to technological processes, including: cooking, baking, boiling, roasting, moist heat, dry heat, fermentation, proteolysis, storage and multiple methods.
(e) These technological processes cause modification of food antigens, which can result in new antigenic determinants and, hence, higher levels of antibody production in blood and saliva.
(f) Comparing the level of IgG, IgM and IgA in blood and IgA, IgM in saliva against unprocessed food versus processed food antigens.
(g) Lower than normal levels or about normal levels of IgG, IgM and IgA against both unprocessed and processed foods indicate optimal conditions.
(h) Higher than normal levels of IgG, IgM and IgA in blood and IgA, IgM in saliva against modified or processed food antigens but not against raw or unmodified food antigens indicate delayed food allergy or food intolerance to modified and processed food antigens.
(i) Measurement of IgG, IgM and IgA in blood and IgA, IgM in saliva against modified or processed food antigens results in enhanced antibody detection and, therefore, a better method of diagnosis for delayed food sensitivities or food allergy and intolerance.

2- The method according to claim 1, wherein the level of antibodies is determined by antigen-antibody reaction methodology such as enzyme-linked immunosorbent assay (ELISA), dot blot, Western blot, radioimmunoassay, agglutination, flow cytometry, proteomic assay and others.

3- The method according to claim 1, wherein the level of antibodies is determined by the antibodies' abilities to bind to antigens prepared from processed or modified foods, including denatured proteins after being purified.

4- The method according to claim 1, wherein the dietary antigen is obtained from a food category selected from the group consisting of milk and products thereof, eggs and products thereof, meat and products thereof, fish, mollusks, and crustaceans and products thereof, oils, fats and products thereof, grains and products thereof, pulses, seeds kernels, nuts and products thereof, vegetables and products thereof, fruits and products thereof, sugar, sugar products, chocolate products and confectionery; and spices and herbs.

5- The method according to claim 1, wherein the dietary antigens are extracted from different foods obtained from grocery stores in their raw and modified forms, for example: apple and apple cider; beer and barley, hops and yeast; hamburger and beef, onion and seasoning; hotdog and beef, pork, turkey, corn, and wheat; sausage and beef, pork and soy; whipped cream and milk, corn, coconut, palm oil and vegetable oil.

6- The method according to claims 4 and 5, wherein the milk and products thereof are selected from American cheese, cheddar cheese, cottage cheese, cow's milk, goat's milk, Swiss cheese, or yoghurt.

7- The method according to claims 4 and 5, wherein the eggs and products thereof are cooked egg and products containing cooked egg such as cake, mayonnaise, potato salad and others.

8- The method according to claims 4 and 5, wherein the meat and products thereof is selected from beef, chicken, pork or turkey and products containing them such as buffalo wings, fried chicken, hamburger, hotdog, sausage and steak.

9- The method according to claims 4 and 5, wherein the fish, mollusks, crustaceans and products thereof are salmon, shrimp, tuna, whitefish and their modified product forms such as canned, fried or baked.

10- The method according to claims 4 and 5, wherein the oils, fats and products thereof are butter, vegetable oil, soybean oil, palm oil and products containing them such as french fries, fried chicken, mayonnaise, tater tots, whipped cream and others.

11- The method according to claims 4 and 5, wherein the grains and products thereof are barley, wheat, corn, oat, rice, and products containing them such as doughnut, cake, cereal, pasta, pizza and others.

12- The method according to claims 4 and 5, wherein the pulses, seeds, kernels, nuts and products thereof are beans, coffee beans, lentils, peanuts, pecans, mustard seed, soybean and products containing them such as chili, coffee, boiled lentils, peanut butter, roasted peanuts, roasted pecans, mustard, tofu and others.

13- The method according to claims 4 and 5, wherein the vegetables and products thereof are corn, tomato, onion, potato, cucumber, spinach, mushroom, broccoli, pumpkin, carrot, celery, beet, parsley, lettuce and products containing them such as vegetarian chili, french fries, ketchup, potato salad, vegetable juice and others.

14- The method according to claims 4 and 5, wherein the fruits and products thereof are apple, orange, cranberry, strawberries, olives, lemon, coconut, grape and products containing them such as apple cider, cranberry sauce, orange chicken, ice cream, wine and others.

15- The method according to claims 4 and 5, wherein the sugar, sugar products, chocolate products and confectionery are sugar, sugar dextrose, cocoa and products containing them such as food coloring, doughnuts, cake, cereal and others.

16- The method according to claims 4 and 5, wherein the spices and herbs are garlic, seasoning, vinegar, mustard, turmeric, paprika, parsley and products containing them such as chili, hamburger, pickles, potato salad and others.
